(19) United States
(12) Patent Application Publication
(10) Pub. No.: US 2006/0177918 A1
(43) Pub. Date: Aug. 10, 2006

(54) Compositions, methods and kits for real-time enzyme assays using charged molecules

(75) Inventor: Hongye Sun, San Mateo, CA (US)

Correspondence Address: DECHERT LLP, P.O. BOX 10004, PALO ALTO, CA, 94303, US

(73) Assignee: Applera Corporation, Foster City, CA (US)

(21) Appl. No.: 11/322,013

(22) Filed: Dec. 29, 2005

Related U.S. Application Data
(60) Provisional application No. 60/641,177, filed on Dec. 30, 2004.

(51) Int. Cl. C12N 9/94

(52) U.S. Cl. 435/186

(57) ABSTRACT

Compositions, methods and kits useful for, among other things, detecting, quantifying and/or characterizing enzymes.

### CROSS REFERENCE TO RELATED APPLICATIONS

This application claims benefit under 35 U.S.C. § 119(e) to application Ser. No. 60/641,177, filed Dec. 30, 2004, the contents of which are incorporated herein by reference.

### BACKGROUND

Enzymes are molecules that increase the rate of chemical reactions. Enzymatic assays for detecting, quantifying and/or characterizing enzyme activity have significant biological, medical and industrial applications. In biological systems, enzymes are involved in synthesis and replication of nucleic acids, modification, and degradation of polypeptides, synthesis of metabolites, and many other functions. In medical testing, enzymes are important indicators of the health or disease of human patients. In industry, enzymes are used for many purposes, such as proteases used in laundry detergents, metabolic enzymes to make specialty chemicals such as amino acids and vitamins, and chirally specific enzymes to prepare enantiomerically pure drugs. Assays using reporter molecules are important tools for studying and detecting enzymes that mediate numerous biological and industrial processes. Although numerous approaches have been developed for assaying enzymes using reporter molecules, there remains a great need to find new assay designs that can be used to inexpensively and conveniently detect and characterize a wide variety of enzymes.

### SUMMARY

Provided herein are compositions, methods and kits useful for, among other things, detecting, quantifying and/or characterizing enzymes or agents of interest. In some embodiments, the composition comprises (i) a hydrophobic molecule comprising a hydrophobic moiety, a dye-moiety and optional charge-moiety, and (ii) one or more charge-balance molecules. The hydrophobic moiety is capable of integrating the hydrophobic molecule into a micelle when included in an aqueous solvent at or above its critical micelle concentration (CMC). The charge-balance molecule acts to promote or encourage micelle formation. While not intending to be bound by any theory of operation, it is believed that the charge-balance molecule comprises sufficient opposite charge from the hydrophobic molecule to promote or encourage micelle formation. In some embodiments, the hydrophobic molecule and/or charge-balance molecule, can each independently of the other, comprise a substrate or putative substrate for enzymes or agents of interest. In some embodiments, the optional charge-moiety comprises an enzyme substrate. In some embodiments, the hydrophobic molecule and the charge-balance molecule both comprise the same substrate. In some embodiments, the hydrophobic molecule and the charge-balance molecule comprise different substrates. Non-limiting examples of enzymes that can act upon the substrate include kinases, phosphatases, sulfatases, peptidases, and carboxylases.

In some embodiments, the dye moiety can be a fluorescent moiety. The fluorescent moiety functions to produce a fluorescent signal when the substrate of the composition is acted upon by an enzyme or agent. Non-limiting examples of suitable fluorescent dyes that can comprise the fluorescent moiety(ies) include xanthene dyes such as fluorescein, sulfofluorescein and rhodamine dyes, cyanine dyes, bodipy dyes and squaraine dyes. Fluorescent moieties comprising other fluorescent dyes may also be used.

In some embodiments, both the hydrophobic molecule and the charge-balance molecule comprise a dye moiety. For example, the hydrophobic molecule can comprise a fluorescent moiety and the charge-balance molecule can comprise a quenching moiety. A quenching moiety can be any moiety capable of quenching the fluorescence of a fluorescent moiety when the quenching moiety is in close proximity to the fluorescent moiety. In some embodiments, the hydrophobic molecule can comprise a quenching moiety and the charge-balance molecule can comprise a fluorescent moiety.

In some embodiments, a quenching moiety can be included into the micelle as a separate quenching molecule. The quenching molecule can include a hydrophobic moiety and a quenching moiety that quenches the light signal of the fluorescent moiety.

In another aspect, a method of detecting and/or characterizing an enzyme activity in a sample is provided. The sample is contacted with a micelle and a fluorescent signal is detected. In some embodiments, the micelle comprises (i) a hydrophobic molecule comprising a hydrophobic moiety, a dye-moiety, and an optional charge-moiety; and (ii) one or more charge-balance molecules. In some embodiments, the hydrophobic molecule and/or charge-balance molecule can independently of the other comprise a substrate or putative substrate for enzymes or agents of interest. In some embodiments, the optional charge-moiety comprises an enzyme substrate. In some embodiments, the hydrophobic molecule and the charge-balance molecule both comprise the same substrate. In some embodiments, the hydrophobic molecule and the charge-balance molecule comprise different substrates.

In another aspect, a kit for use in detecting and/or characterizing an enzyme activity in a sample is provided. In some embodiments, the kit comprises (i) a hydrophobic molecule comprising a hydrophobic moiety, a dye moiety and an optional charge-moiety, and (ii) one or more charge-balance molecules. In some embodiments, the hydrophobic molecule and/or charge-balance molecule can independently of the other comprise a substrate or putative substrate for enzymes or agents of interest. In some embodiments, the optional charge-moiety comprises an enzyme substrate. In some embodiments, the hydrophobic molecule and the charge-balance molecule both comprise the same substrate. In some embodiments, the hydrophobic molecule and the charge-balance molecule comprise different substrates. These and other features of the present teachings are set forth below.

### BRIEF DESCRIPTION OF THE FIGURES

The skilled artisan will understand that the drawings, described below, are for illustration purposes only. The drawings are not intended to limit the scope of the present teaching in anyway.

FIGS. 1A-B show electron micrographs of micelles comprising a hydrophobic molecule, C₁₇OOOK(tet)RQGSFRA-amide (FIG. 1A) and a phosphorylated hydrophobic molecule, C₁₇OOOK(tet)RQGS(p)FRA-amide (FIG. 1B); the bar represents 100 nm.

FIG. 2 depicts the effect of varying concentrations (0, 2.5, 5, 10, 20, 50 μM) of myelin basic protein (MBP) on quenching the fluorescence of a hydrophobic molecule, C₁₆OOOK(Dye2)EEIYGEF-amide (10 μM) in 20 mM Tris (pH 7.6) and 5 mM MgCl₂.

FIGS. 3A-C show the rate of reaction of PKCβII (FIG. 3A), MAP Kinase1/Erk1 (FIG. 3B), and MAP Kinase2/Erk2 (FIG. 3C) against MPB (10 μM) with the hydrophobic molecule, C₁₆OOOK(Dye2)EEIYGEF-amide (10 μM) in 25 mM Tris (pH 7.6), 5 mM MgCl₂, with 0 and 500 μM ATP.

FIGS. 4A-C show the apparent Kₘ^{ATP} of PKCβII (FIG. 4A), MAP Kinase1/Erk1 (FIG. 4B), and MAP Kinase2/Erk2 (FIG. 4C) with MPB (10 μM) and the hydrophobic molecule, C₁₆OOOK(Dye2)EEIYGEF-amide (10 μM) in 25 mM Tris (pH 7.6), and 5 mM MgCl₂.

FIGS. 5A-B show staurosporine (FIG. 5A) and H89 (FIG. 5B) inhibition of PKCβII with MPB (10 μM) and the hydrophobic molecule, C₁₆OOOK(Dye2) EEIYGEF-amide (10 μM) in 25 mM Tris (pH 7.6), 5 mM MgCl₂.

### DETAILED DESCRIPTION

It is to be understood that both the foregoing summary and the following description of various embodiments are exemplary and explanatory only and are not restrictive of the present teachings. In this application, the use of the singular includes the plural unless specifically stated otherwise. Also, the use of “or” means “and/or” unless stated otherwise. Similarly, “comprise,” “comprises,” “comprising,” “include,” “includes” and “including” are not intended to be limiting.

5.1 Definitions

As used herein, the following terms are intended to have the following meanings:

“Detect” and “detection” have their standard meaning, and are intended to encompass detection, measurement, and characterization of a selected enzyme or enzyme activity. For example, enzyme activity can be “detected” in the course of detecting, screening for, or characterizing inhibitors, activators, and modulators of the enzyme activity.

“Fatty Acid” has its standard meaning and is intended to refer to a long-chain hydrocarbon carboxylic acid in which the hydrocarbon chain is saturated, mono-unsaturated or polyunsaturated. The hydrocarbon chain can be linear, branched or cyclic, or can comprise a combination of these features, and can be unsubstituted or substituted. Fatty acids typically have the structural formula RC(O)OH, where R is a substituted or unsubstituted, saturated, mono-unsaturated or polyunsaturated hydrocarbon comprising from 6 to 30 carbon atoms which has a structure that is linear, branched, cyclic or a combination thereof.

“Micelle” has its standard meaning and is intended to refer to an aggregate formed by amphipathic molecules in water or an aqueous environment such that their polar ends or portions are in contact with the water or aqueous environment and their nonpolar ends or portions are in the interior of the aggregate. A micelle can take any shape or form, including but not limited to, a non-lamellar “detergent-like” aggregate that does not enclose a portion of the water or aqueous environment, or a unilamellar or multilamellar “vesicle-like” aggregate that encloses a portion of the water or aqueous environment, such as, for example, a liposome.

“Quench” has its standard meaning and is intended to refer to a reduction in the fluorescence intensity of a fluorescent group or moiety as measured at a specified wavelength, regardless of the mechanism by which the reduction is achieved. As specific examples, the quenching can be due to molecular collision, energy transfer such as FRET, photoinduced electron transfer such as PET, a change in the fluorescence spectrum (color) of the fluorescent group or moiety or any other mechanism (or combination of mechanisms). The amount of the reduction is not critical and can vary over a broad range. The only requirement is that the reduction be detectable by the detection system being used. Thus, a fluorescence signal is “quenched” if its intensity at a specified wavelength is reduced by any measurable amount. A fluorescence signal is “substantially quenched” if its intensity at a specified wavelength is reduced by at least 50%, for example by 50%, 60%, 70%, 75%, 80%, 85%, 90%, 95%, 96%, 97%, 98%, 99% or even 100%.

Polypeptide sequences are provided with an orientation (left to right) of the N terminus to C terminus, with amino acid residues represented by the standard 3-letter or 1-letter codes (e.g., Stryer, L., Biochemistry, 2nd Ed., W.H. Freeman and Co., San Francisco, Calif., page 16 (1981)).

“Polynucleotides or Oligonucleotides” refer to nucleobase polymers or oligomers in which the nucleobases are connected by sugar phosphate linkages (sugar-phosphate backbone). Exemplary poly- and oligonucleotides include polymers of 2′ deoxyribonucleotides (DNA) and polymers of ribonucleotides (RNA). A polynucleotide may be composed entirely of ribonucleotides, entirely of 2′ deoxyribonucleotides or combinations thereof.

“Polynucleotide or Oligonucleotide Analog” refers to nucleobase polymers or oligomers in which the nucleobases are connected by a sugar phosphate backbone comprising one or more sugar phosphate analogs. Typical sugar phosphate analogs include, but are not limited to, sugar alkylphosphonates, sugar phosphoramidites, sugar alkyl- or substituted alkylphosphotriesters, sugar phosphorothioates, sugar phosphorodithioates, sugar phosphates and sugar phosphate analogs in which the sugar is other than 2′-deoxyribose or ribose, nucleobase polymers having positively charged sugar-guanidyl interlinkages such as those described in U.S. Pat. No. 6,013,785 and U.S. Pat. No. 5,696,253 (see also, Dagani 1995, Chem. & Eng. News 4-5:1153; Dempey et al., 1995, J. Am. Chem. Soc. 117:6140-6141). Such positively charged analogues in which the sugar is 2′-deoxyribose are referred to as “DNGs,” whereas those in which the sugar is ribose are referred to as “RNGs.” Specifically included within the definition of poly- and oligonucleotide analogs are locked nucleic acids (LNAs; see, e.g. Elayadi et al., 2002, Biochemistry 41:9973-9981; Koshkin et al., 1998, J. Am. Chem. Soc. 120:13252-3; Koshkin et al., 1998, Tetrahedron Letters, 39:4381-4384; Jumar et al., 1998, Bioorganic & Medicinal Chemistry Letters 8:2219-2222; Singh and Wengel, 1998, Chem. Commun., 12:1247-1248; WO 00/56746; WO 02/28875; and, WO 01/48190; all of which are incorporated herein by reference in their entireties).

“Polynucleotide or Oligonucleotide Mimic” refers to a nucleobase polymer or oligomers in which one or more of the backbone sugar-phosphate linkages is replaced with a sugar-phosphate analog. Such mimics are capable of hybridizing to complementary polynucleotides or oligonucleotides, or polynucleotide or oligonucleotide analogs or to other polynucleotide or oligonucleotide mimics, and may include backbones comprising one or more of the following linkages: positively charged polyamide backbone with alkylamine side chains as described in U.S. Pat. No. 5,786,461; U.S. Pat. No. 5,766,855; U.S. Pat. No. 5,719,262; U.S. Pat. No. 5,539,082 and WO 98/03542 (see also, Haaima et al., 1996, Angewandte Chemie Int'l Ed. in English 35:1939-1942; Lesnick et al., 1997, Nucleosid. Nucleotid. 16:1775-1779; D'Costa et al., 1999, Org. Lett. 1:1513-1516 see also Nielsen, 1999, Curr. Opin. Biotechnol. 10:71-75); uncharged polyamide backbones as described in WO 92/20702 and U.S. Pat. No. 5,539,082; uncharged morpholino-phosphoramidate backbones as described in U.S. Pat. No. 5,698,685, U.S. Pat. No. 5,470,974, U.S. Pat. No. 5,378,841 and U.S. Pat. No. 5,185,144 (see also, Wages et al., 1997, BioTechniques 23:1116-1121); peptide-based nucleic acid mimic backbones (see, e.g., U.S. Pat. No. 5,698,685); carbamate backbones (see, e.g., Stirchak & Summerton, 1987, J. Org. Chem. 52:4202); amide backbones (see, e.g., Lebreton, 1994, Synlett. February, 1994:137); methylhydroxyl amine backbones (see, e.g., Vasseur et al., 1992, J. Am. Chem. Soc. 114:4006); 3′-thioformacetal backbones (see, e.g., Jones et al., 1993, J. Org. Chem. 58:2983) and sulfamate backbones (see, e.g., U.S. Pat. No. 5,470,967). All of the preceding references are herein incorporated by reference.

“Peptide Nucleic Acid” or “PNA” refers to poly- or oligonucleotide mimics in which the nucleobases are connected by amino linkages (uncharged polyamide backbone) such as described in any one or more of U.S. Pat. Nos. 5,539,082, 5,527,675, 5,623,049, 5,714,331, 5,718,262, 5,736,336, 5,773,571, 5,766,855, 5,786,461, 5,837,459, 5,891,625, 5,972,610, 5,986,053, 6,107,470, 6,451,968, 6,441,130, 6,414,112 and 6,403,763; all of which are incorporated herein by reference. The term “peptide nucleic acid” or “PNA” shall also apply to any oligomer or polymer comprising two or more subunits of those polynucleotide mimics described in the following publications: Lagriffoul et al., 1994, Bioorganic & Medicinal Chemistry Letters, 4: 1081-1082; Petersen et al., 1996, Bioorganic & Medicinal Chemistry Letters, 6: 793-796; Diderichsen et al, 1996, Tett. Lett. 37: 475-478; Fujii et al., 1997, Bioorg. Med. Chem. Lett. 7: 637-627; Jordan et al., 1997, Bioorg. Med. Chem. Lett. 7: 687-690; Krotz et al., 1995, Tett. Lett. 36: 6941-6944; Lagriffoul et al, 1994, Bioorg. Med. Chem. Lett. 4: 1081-1082; Diederichsen, U., 1997, Bioorganic & Medicinal Chemistry 25 Letters, 7: 1743-1746; Lowe et al., 1997, J. Chem. Soc. Perkin Trans. 1, 1: 539-546; Lowe et al., 1997. J. Chem. Soc. Perkin Trans. 11: 547-554; Lowe et al., 1997, I. Chem. Soc. Perkin Trans. 1 1:5 55-560; Howarth et al., 1997, I. Org. Chem. 62: 5441-5450; Altmann, K-H et al., 1997, Bioorganic & Medicinal Chemistry Letters, 7: 1119-1122; Diederichsen, U., 1998, Bioorganic & Med. Chem. Lett., 8:165-168; Diederichsen et al., 1998, Angew. Chem. mt. Ed., 37: 302-305; Cantin et al., 1997, Tett. Lett., 38: 4211-4214; Ciapetti et al., 1997, Tetrahedron, 53: 1167-1176; Lagriffoule et al., 1997, Chem. Eur. 1.′3: 912-919; Kumar et al., 2001, Organic Letters 3(9): 1269-1272; and the Peptide-Based Nucleic Acid Mimics (PENAMs) of Shah et al. as disclosed in WO 96/04000. All of which are incorporated herein by reference.

5.2 Compositions

Provided herein are compositions, methods and kits useful for, among other things, detecting, quantifying and/or characterizing enzymes. The compositions generally comprise a hydrophobic molecule and one or more charge-balance molecules. In some embodiments, the hydrophobic molecule comprises one or more charged chemical groups, the presence of these groups can discourage or inhibit micelle formation. In some embodiments, the charge-balance molecule comprises chemical groups that have the opposite charge of the chemical groups comprising the hydrophobic molecule, the presence of these groups can act to promote or encourage micelle formation.

In some embodiments, the hydrophobic molecule comprises a hydrophobic moiety, a dye moiety, and an optional charge-moiety. The hydrophobic moiety is capable of integrating the hydrophobic molecule into a micelle when included in an aqueous solvent at or above its critical micelle concentration. In some embodiments, the dye moiety can be a fluorescent moiety and functions to produce a fluorescent signal. While not intending to be bound by any theory of operation, it is believed that the charge-balance molecule comprises sufficient opposite charge from the hydrophobic molecule to promote or encourage micelle formation, whereby the fluorescent moiety is integrated into a micelle and its signal can be quenched. The hydrophobic moiety, dye moiety, and optional charge-moiety can be connected to each other in any way that permits them to perform their respective functions.

The hydrophobic molecule and/or the charge-balance molecule comprise at least one substrate or putative substrate for enzymes or agents of interest. In some embodiments, the optional charge-moiety comprises an enzyme substrate. For example, the hydrophobic molecule and/or the charge-balance molecule can each independently comprise an enzyme substrate. In some embodiments, the hydrophobic molecule and the charge-balance molecule both comprise the same substrate. In some embodiments, the hydrophobic molecule and the charge-balance molecule comprise different substrates. The substrate can be acted upon by an enzyme or agent and/or multiple enzymes or agents. When the substrate is acted upon by an enzyme or agent it can promote the dissociation of the dye moiety from the micelle, thereby reducing or eliminating the quenching effect caused by the interactions between the dye moiety and the micelle. The dissociation can be caused by cleavage of the enzyme recognition site or by the addition, deletion, or substitution of chemical groups, such as charged groups, which can destabilize the micelle, promoting release of the dye moiety therefrom. Release of the dye moiety from the micelle reduces or eliminates the quenching effect, thereby producing a detectable increase in a light signal.

In some embodiments, both the hydrophobic molecule and the charge-balance molecule comprise a dye moiety. In some embodiments, the hydrophobic molecule can comprise a fluorescent moiety and the charge-balance molecule can comprise a quenching moiety. A quenching moiety can be any moiety capable of quenching the fluorescence of a fluorescent moiety when the quenching moiety is in close proximity to the fluorescent moiety. In some embodiments, the quenching moiety can be included into the micelle as a separate quenching molecule. In some embodiments, the hydrophobic molecule can comprise a quenching moiety and the charge-balance molecule can comprise a fluorescent moiety.

5.3 Hydrophobic Moiety

The hydrophobic moiety acts to anchor or integrate the various molecules described herein into the micelle. The exact numbers, lengths, size and/or compositions of the hydrophobic moiety can be varied. For example, in embodiments employing two or more hydrophobic moieties, each hydrophobic moiety may be the same, or some or all of the hydrophobic moieties may differ. As a specific example, in some embodiments, the hydrophobic molecule and the charge-balance molecule, each can comprise a hydrophobic moiety. The two hydrophobic moieties can be the same or they can differ from another. In some embodiments, the hydrophobic moiety(ies) of the hydrophobic molecule can be the same length, size and/or composition as the hydrophobic moiety(ies) of the charge-balance molecule. In some embodiments, the hydrophobic moiety(ies) of the hydrophobic molecule can differ in length, size and/or composition from the hydrophobic moiety(ies) of the charge-balance molecule.

As another specific example, in some embodiments, the hydrophobic molecule can comprise two hydrophobic moieties. The two hydrophobic moieties can be the same or they can differ from another. In some embodiments, the hydrophobic moieties can be the same length, size and/or composition. In some embodiments, the hydrophobic moieties may differ in length, size and/or composition. Additional exemplary embodiments of molecules comprising two hydrophobic moieties are described in U.S. application Ser. No. 10/997,066 entitled “Ligand-containing micelles and uses thereof”, filed on Nov. 24, 2004, the disclosure of which is incorporated herein by reference.

In some embodiments, the hydrophobic moiety comprises a substituted or unsubstituted hydrocarbon of sufficient hydrophobic character (e.g., length and/or size) to cause the hydrophobic molecule and/or the charge-balance molecule to become integrated or incorporated into a micelle when the molecule(s) is placed in an aqueous environment at a concentration above a micelle-forming threshold, such as at or above its critical micelle concentration (CMC). In other embodiments, the hydrophobic moieties comprise a substituted or unsubstituted hydrocarbon comprising from 6 to 30 carbon atoms, or from 6 to 25 carbon atoms, or from 6 to 20 carbon atoms, or from 6 to 15 carbon atoms, or from 8 to 30 carbon atoms, or from 8 to 25 carbon atoms, or from 8 to 20 carbon atoms, or from 8 to 15 carbon atoms, or from 12 to 30 carbon atoms, or from 12 to 25 carbon atoms, or from 12 to 20 carbon atoms. The hydrocarbon can be linear, branched, cyclic, or any combination thereof, and can optionally include one or more of the same or different substituents. Exemplary linear hydrocarbon groups comprise C6, C7, C8, C9, C10, C11, C12, C13, C14, C15, C16, C17, C18, C19, C20, C22, C24, and C26 alkyl chains.

In some embodiments, the hydrophobic moiety is fully saturated. In some embodiments, the hydrophobic moiety can comprise one or more carbon-carbon double bonds which can be, independently of one another, in the cis or trans configuration, and/or one or more carbon-carbon triple bonds. In some cases, the hydrophobic moiety can have one or more cycloalkyl groups, or one or more aryl rings or arylalkyl groups, such as one or two phenyl rings.

In some embodiments, the hydrophobic moiety is a nonaromatic moiety that does not have a cyclic aromatic pi electron system. In some embodiments, if the hydrophobic moiety contains one or more unsaturated carbon-carbon bonds, those carbon-carbon bonds are not conjugated. In another embodiment, the structure of the hydrophobic moiety is incapable of interacting with the fluorescent moiety, by a FRET or stacking interaction, to quench fluorescence of the fluorescent moiety. Also encompassed herein are embodiments that involve a combination of any two or more of the foregoing embodiments. Optimization testing can be done by making several hydrophobic and/or charge-balance molecules having different hydrophobic moieties.

In some embodiments, the molecule(s) of the composition comprises two hydrophobic moieties linked to the C1 and C2 carbons of a glycerolyl group via ester linkages (or other linkages). The two hydrophobic moieties can be the same or they can differ from another. In a specific embodiment, each hydrophobic moiety is selected to correspond to the hydrocarbon chain or “tail” of a naturally occurring fatty acid. In another specific embodiment, the hydrophobic moieties are selected to correspond to the hydrocarbon chains or tails of a naturally occurring phospholipid. Non-limiting examples of hydrocarbon chains or tails of commonly occurring fatty acids are provided in Table 1, below:

TABLE 1

| Length:Number of Unsaturations | Common Name |
|---|---|
| 14:0 | myristic acid |
| 16:0 | palmitic acid |
| 18:0 | stearic acid |
| 18:1 cisΔ⁹ | oleic acid |
| 18:2 cisΔ^{9,12} | linoleic acid |
| 18:3 cisΔ^{9,12,15} | linonenic acid |
| 20:4 cisΔ^{5,8,11,14} | arachidonic acid |
| 20:5 cisΔ^{5,8,11,14,17} | eicosapentaenoic acid |
| | (an omega-3 fatty acid) |

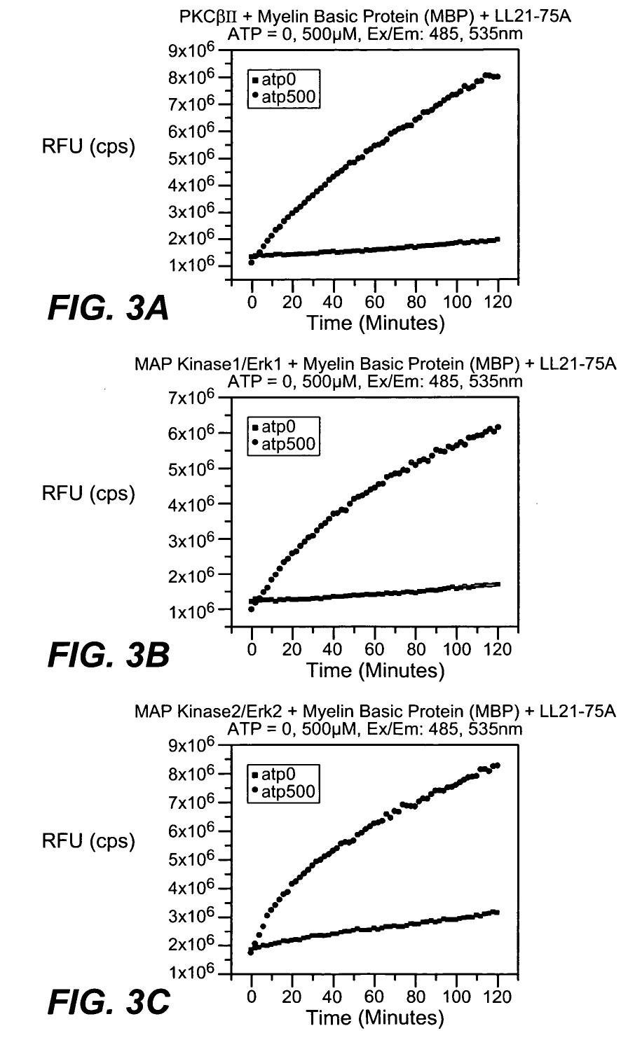

In some embodiments, the hydrophobic moiety comprises amino acids or amino acid analogs that have hydrophobic side chains. The amino acids or analogs are chosen to provide sufficient hydrophobicity to integrate the molecule(s) of the composition into a micelle under the assay conditions used to detect the enzymes. Exemplary hydrophobic amino acids include alanine, glycine, valine, leucine, isoleucine, proline, phenylalanine, methionine, tryptophan, and cysteine as described in Alberts, B., et al., Molecular Biology of the Cell, 4^{th} Ed., Garland Science, New York, N.Y., FIG. 3.3 (2002)). Exemplary amino acid analogs include norvaline, aminobutyric acid, cyclohexylalanine, butylglycine, phenylglycine, and N-methylvaline (see “Amino Acids and Amino Acid Analogs” section 2002-2003 Novabiochem catalog).

The hydrophobicity of a polypeptide can be calculated by assigning each amino acid a hydrophobicity value and then averaging the values along the polypeptide chain. Hydrophobicity values for the common amino acids are shown Table 2.

TABLE 2
Hydrophobicity of Amino Acids

| | Monera et al.¹ | Hopp-Woods² | Kyte-Doolittle³ |
|---|---|---|---|
| Amino Acid | Hydrophobicity at | Hydrophobicity | Hydrophobicity |
| (IUPAC) | pH 7 | scale | scale |
| Alanine (A) | 41 | −0.5 | −1.8 |
| Cysteine (C) | 49 | −1.0 | −2.5 |
| Aspartic acid (D) | −55 | 3.0 | 3.5 |
| Glutamic acid (E) | −31 | 3.0 | 3.5 |
| Phenylalanine (F) | 100 | −2.5 | −2.8 |
| Glycine (G) | 0 | 0.0 | 0.4 |
| Histidine (H) | 8 | −0.5 | 3.2 |
| Isoleucine (I) | 99 | −1.8 | −4.5 |
| Lysine (K) | −23 | 3.0 | 3.9 |
| Leucine (L) | 97 | −1.8 | −3.8 |
| Methionine (M) | 74 | −1.3 | −1.9 |
| Asparagine (N) | −28 | 0.2 | 3.5 |
| Proline (P) | −46 (pH 2) | 0.0 | 1.6 |
| Glutamine (Q) | −10 | 0.2 | 3.5 |
| Arginine (R) | −14 | 3.0 | 4.5 |
| Serine (S) | −5 | 0.3 | 0.8 |
| Threonine (T) | 13 | −0.4 | 0.7 |
| Valine (V) | 76 | −1.5 | −4.2 |
| Tryptophan (W) | 97 | −3.4 | 0.9 |
| Tyrosine (Y) | 63 | −2.3 | 1.3 |

¹Monera et al. J. Protein Sci 1: 219-329 (1995) (The values were normalized so that the most hydrophobic residue (phenylalanine) is given a value of 100 relative to glycine, which is considered neutral (0 value)).
²Hoop TP and Woods KR: Prediction of protein antigenic determinants from amino acid sequences. Proc Natl Acad Sci USA 78: 3824, 1981.
³Kyte J and Doolittle RF: A simple method for displaying the hydropathic character of a protien. J Mol Biol 157: 105, 1982.

The exact number of amino acids or amino acid analogs chosen will vary depending on the sequence of the amino acids selected and the presence of other constituents. In some embodiments, the hydrophobic moiety comprises the same amino acid or amino acid analog. For example, the hydrophobic moiety can comprise poly(leucine) from 1 and 10 leucine residues. In some embodiments, the hydrophobic moiety comprises a mixture of amino acids or amino acid analogs. For example, the hydrophobic moiety can comprise a mixture of amino acids, such as leucine and isoleucine, from 1 to 10 leucine resides and from 1 to 10 isoleucine residues can be used.

In some embodiments, the hydrophobic moiety can comprise a mixture of amino acids, amino acid analogs, and hydrocarbons. For example, in some embodiments, the hydrophobic moiety can comprise from 1 to 10 residues of amino acids or amino acid analogs and a hydrocarbon comprising from 2 to 30 carbons atoms.

The hydrophobic moiety can be connected to the other moieties comprising the hydrophobic molecule and/or the charge-balance molecule in any way that permits them to perform their respective functions. For example, if the hydrophobic molecule comprises a hydrophobic moiety, a dye moiety, and a charge-moiety, the moieties can be connected directly to one another, i.e., covalently linked to each other. In other embodiments, one, some, or all of the moieties can be connected indirectly to one another, i.e., via one or more optional linkers.

For embodiments of molecule(s) of the compositions in which the hydrophobic moiety is linked to the dye moiety (discussed below), it will be understood that the hydrophobic moiety is distinct from the dye moiety because the hydrophobic moiety does not comprise any of the atoms in the dye moiety that are part of the aromatic or conjugated pi-electron system that produces the fluorescent signal. Thus, if a hydrophobic moiety is connected to the C4 position of a xanthene ring (e.g., the C4′ position of a fluorescein or rhodamine dye), the hydrophobic moiety does not comprise any of the aromatic ring atoms of the xanthene ring

5.4 Dye Moiety

The compositions described herein comprise at least one dye moiety. In some embodiments, the dye moiety comprises a fluorescent moiety which can be selectively “turned on” when the enzyme substrate is modified as described herein. The fluorescent moiety can comprise any entity that provides a fluorescent signal and that can be used in accordance with the methods and principles described herein.

In some embodiments, the dye moiety comprises a quenching moiety. The quenching moiety can be any moiety capable of quenching the fluorescence of a fluorescent moiety when the quenching moiety is in close proximity to the fluorescent moiety. Quenching of the fluorescent moiety within the micelle can be achieved in a variety of different ways. In one embodiment, the quenching effect may be achieved or caused by “self-quenching.” Self-quenching can occur when the molecules comprising the fluorescent moiety are present in the micelle at a concentration sufficient or molar ratio high enough to bring their fluorescent moieties in close enough proximity to one another such that their fluorescence signals are quenched. Release of the fluorescent moieties from the micelle reduces or abolishes the “self-quenching,” producing an increase in their fluorescence signals. As used herein, a fluorescent moiety is “released” or “removed” from a micelle if any molecule or molecular fragment that contains the fluorescent moiety is released or removed from the micelle.

In some embodiments, the hydrophobic molecule comprises at least one dye moiety. In some embodiments, the hydrophobic molecule comprises a fluorescent moiety. In some embodiments, the hydrophobic molecule comprises two dye moieties capable of self-quenching.

In some embodiments, the charge-balance molecule comprises at least one dye moiety. In some embodiments, the charge-balance molecule comprises a fluorescent moiety. In some embodiments, the charge-balance molecule comprises two dye moieties capable of self-quenching.

In some embodiments, the hydrophobic molecule and the charge-balance molecule can each comprises at least one dye moiety. In some embodiments, the hydrophobic molecule and the charge-balance molecule can each comprise the same dye moiety. In some embodiments, the hydrophobic molecule and the charge-balance molecule can each comprise a different dye moiety. In some embodiments, one molecule comprises a quenching moiety and one molecule comprises a fluorescent moiety. In some embodiments, the hydrophobic molecule comprises a dye moiety and the charge-balance molecule comprises a dye moiety capable of self-quenching. In some embodiments, the charge-balance molecule comprises a dye moiety and the hydrophobic molecule comprises a dye moiety capable of self-quenching.

In some embodiments, the quenching moiety can be included as a separate quenching molecule. The quenching molecule can include a hydrophobic moiety and a quenching moiety that quenches the light signal of the dye moiety. The quenching moiety can be positioned so that it is able to intramolecularly quench the fluorescence of the dye moiety on the hydrophobic molecule and/or the charge-balance molecule, which includes it, or, alternatively, the quenching moiety may be positioned so that intramolecular quenching does not occur. In either embodiment, the quenching moiety may intermolecularly quench the fluorescence of a dye moiety on another molecule in the micelle which is in close proximity thereto. When the substrate is acted upon by a specified enzyme it “deactivates” the quenching effect by relieving the close proximity of the quenching and fluorescent moieties, thereby generating a measurable increase in fluorescence signals.

The dye moiety can be connected to the molecules described herein in any way that permits them to perform their respective functions. For example, if the hydrophobic molecule comprises a hydrophobic moiety and a dye moiety, the moieties can be connected directly to one another, i.e., covalently linked to each other. In other embodiments, one, some or all of the moieties can be connected indirectly to one another, i.e., via one or more optional linkers.

As another specific example, if the hydrophobic molecule comprises a hydrophobic moiety, a dye moiety, and a charge-moiety, the moieties can be connected directly to one another, i.e., covalently linked to each other. In other embodiments, one, some or all of the moieties can be connected indirectly to one another, i.e., via one or more optional linkers.

For any given assay, the fluorescent moiety can be soluble or insoluble. For example, in some embodiments the fluorescent moiety is soluble under conditions of the assay so as to facilitate removal of the released fluorescent moiety from the micelle into the assay medium. In other embodiments, provided that self-quenching does not occur, the fluorescent moiety is insoluble under conditions of the assay so that the fluorescent moiety can precipitate out of solution and localize at the site at which it was produced, thereby producing an increase in the fluorescent signal as compared to the signal observed in solution.

The quenching effect can be achieved or caused by other moieties comprising the micelle. These moieties are referred to as “quenching moieties,” regardless of the mechanism by which the quenching is achieved. Such quenching moieties and quenching molecules are described in more detail, below. By modifying the quenching moieties to reduce or eliminate their quenching effects, or by removing the fluorescent moiety from proximity of the quenching moieties, the fluorescence of the fluorescent moiety can be substantially restored. Any mechanism that is capable of causing quenching or changes in fluorescence properties may be used in the micelles and methods described herein.

The degree of quenching achieved within the micelle is not critical for success, provided that it is measurable by the detection system being used. As will be appreciated, higher degrees of quenching are desirable, because the greater the quenching effect, the lower the background fluorescence prior to removal of the quenching effect. In theory, a quenching effect of 100%, which corresponds to complete suppression of a measurable fluorescence signal, would be ideal. In practice, any measurable amount will suffice. The amount and/or molar percentage of hydrophobic molecule and/or the charge-balance molecule and optional quenching molecule in a micelle necessary to provide a desired degree of quenching in the micelle may vary depending upon, among other factors, the choice of the fluorescent moiety. The amount and/or molar percentage of any hydrophobic molecule and/or the charge-balance molecule and optional quenching molecule (or mixture of optional quenching molecules) contained in a micelle in order to obtain a sufficient degree of quenching can be determined empirically.

Typically, the dye moiety of the hydrophobic molecule and/or the charge-balance molecule comprises a fluorescent dye that in turn comprises a resonance-delocalized system or aromatic ring system that absorbs light at a first wavelength and emits fluorescent light at a second wavelength in response to the absorption event. A wide variety of such fluorescent dye molecules are known in the art. For example, fluorescent dyes can be selected from any of a variety of classes of fluorescent compounds, such as xanthenes, rhodamines, fluoresceins, cyanines, phthalocyanines, squaraines, bodipy dyes, coumarins, oxazines, and carbopyronines.

In some embodiments, the fluorescent dye comprises a xanthene dye. Generally, xanthene dyes are characterized by three main features: (1) a parent xanthene ring; (2) an exocyclic hydroxyl or amine substituent; and (3) an exocyclic oxo or imminium substituent. The exocyclic substituents are typically positioned at the C3 and C6 carbons of the parent xanthene ring, although “extended” xanthenes in which the parent xanthene ring comprises a benzo group fused to either or both of the C5/C6 and C3/C4 carbons are also known. In these extended xanthenes, the characteristic exocyclic substituents are positioned at the corresponding positions of the extended xanthene ring. Thus, as used herein, a “xanthene dye” generally comprises one of the following parent rings:

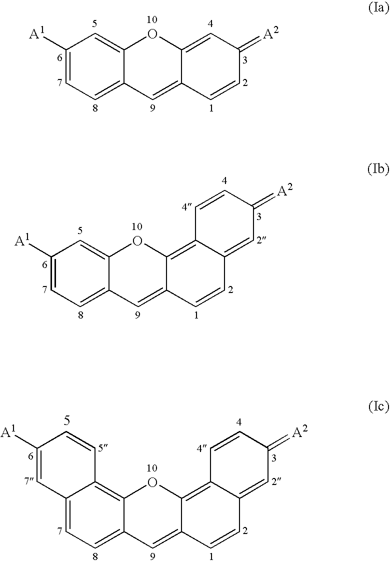

In the parent rings depicted above, A¹ is OH or NH₂ and A² is O or NH₂⁺. When A¹ is OH and A² is O, the parent ring is a fluorescein-type xanthene ring. When A¹ is NH₂ and A² is NH₂⁺, the parent ring is a rhodamine-type xanthene ring. When A¹ is NH₂ and A² is O, the parent ring is a rhodol-type xanthene ring.

One or both of nitrogens of A¹ and A² (when present) and/or one or more of the carbon atoms at positions C1, C2, C2″, C4, C4″, C5, C5″, C7″, C7 and C8 can be independently substituted with a wide variety of the same or different substituents. In one embodiment, typical substituents comprise, but are not limited to, —X, —Ra, —ORa, —SRa, —NR^{a}R^{a}, perhalo (C₁-C₆) alkyl, —CX₃, —CF₃, —CN, —OCN, —SCN, —NCO, —NCS, —NO, —NO₂, —N₃, —S(O)₂O⁻, —S(O)₂OH, —S(O)₂R^{a}, —C(O)R, —C(O)X, —C(S)R^{a}, —C(S)X, —C(O)ORa, —C(O)O⁻, —C(S)ORa, —C(O)SRa, —C(S)SRa, —C(O)NR^{a}R^{a}, —C(S)NR^{a}R^{a} and —C(NR)NR^{a}R^{a}, where each X is independently a halogen (preferably —F or —Cl) and each R^{a} is independently hydrogen, (C₁-C₆)alkyl, (C₁-C₆)alkanyl, (C₁-C₆)alkenyl, (C₁-C₆)alkynyl, (C₅-C₂₀)aryl, (C₆-C₂₆)arylalkyl, (C₅-C₂₀)arylaryl, 5-20 membered heteroaryl, 6-26 membered heteroarylalkyl, 5-20 membered heteroaryl-heteroaryl, carboxyl, acetyl, sulfonyl, sulfinyl, sulfone, phosphate, or phosphonate. Generally, substituents which do not tend to completely quench the fluorescence of the parent ring are preferred, but in some embodiments quenching substituents may be desirable. Substituents that tend to quench fluorescence of parent xanthene rings are electron-withdrawing groups, such as —NO₂, —Br and —I.

The C1 and C2 substituents and/or the C7 and C8 substituents can be taken together to form substituted or unsubstituted buta[1,3]dieno or (C₅-C₂₀)aryleno bridges. For purposes of illustration, exemplary parent xanthene rings including unsubstituted benzo bridges fused to the C1/C2 and C7/C8 carbons are illustrated below:

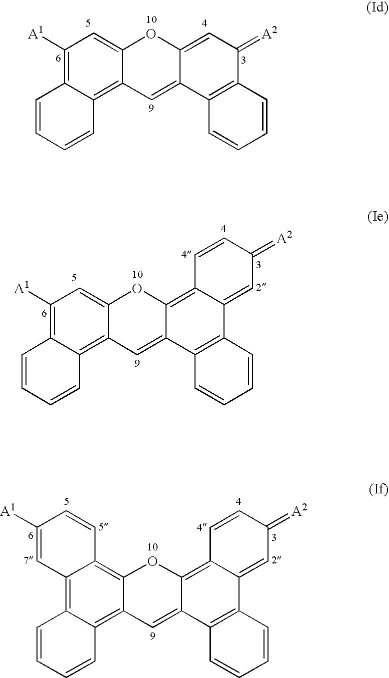

The benzo or aryleno bridges may be substituted at one or more positions with a variety of different substituent groups, such as the substituent groups previously described above for carbons C1-C8 in structures (Ia)-(Ic), supra. In embodiments including a plurality of substituents, the substituents may all be the same, or some or all of the substituents can differ from one another.

When A¹ is NH₂ and/or A² is NH₂⁺, the nitrogen atoms may be included in one or two bridges involving adjacent carbon atom(s). The bridging groups may be the same or different, and are typically selected from (C₁-C₁₂)alkyldiyl, (C₁-C₁₂)alkyleno, 2-12 membered heteroalkyldiyl and/or 2-12 membered heteroalkyleno bridges. Non-limiting exemplary parent rings that comprise bridges involving the exocyclic nitrogens are illustrated below:

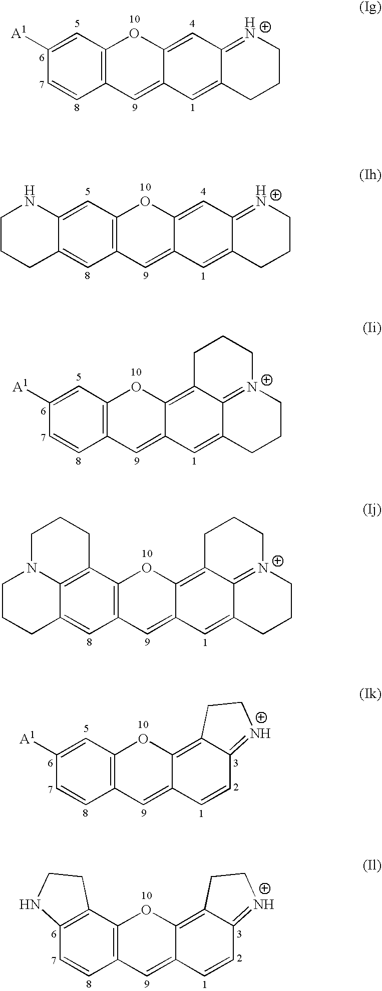

The parent ring may also comprise a substituent at the C9 position. In some embodiments, the C9 substituent is selected from acetylene, lower (e.g., from 1 to 6 carbon atoms) alkanyl, lower alkenyl, cyano, aryl, phenyl, heteroaryl, electron-rich heteroaryl and substituted forms of any of the preceding groups. In embodiments in which the parent ring comprises benzo or aryleno bridges fused to the C1/C2 and C7/C8 positions, such as, for example, rings (Id), (le) and (If) illustrated above, the C9 carbon is preferably unsubstituted.

In some embodiments, the C9 substituent is a substituted or unsubstituted phenyl ring such that the xanthene dye comprises one of the following structures:

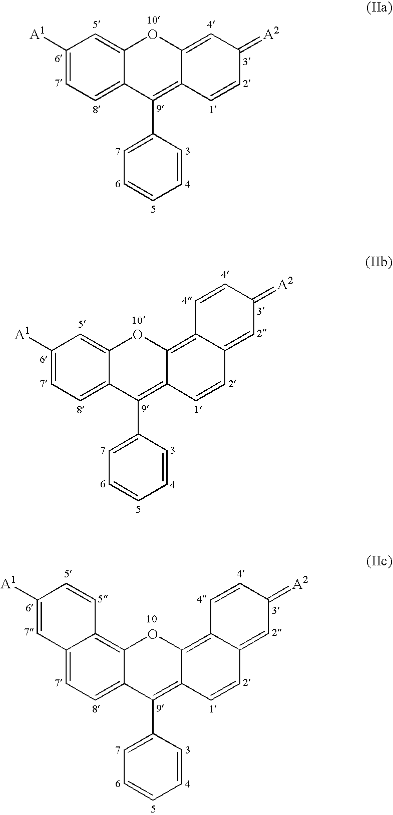

The carbons at positions 3, 4, 5, 6 and 7 may be substituted with a variety of different substituent groups, such as the substituent groups previously described for carbons C₁-C₈. In some embodiments, the carbon at position C3 is substituted with a carboxyl (—COOH) or sulfuric acid (—SO₃H) group, or an anion thereof. Dyes of formulae (IIa), (IIb) and (IIc) in which A¹ is OH and A² is 0 are referred to herein as fluorescein dyes; dyes of formulae (IIa), (IIb) and (IIc) in which A¹ is NH₂ and A² is NH₂⁺ are referred to herein as rhodamine dyes; and dyes of formulae (IIa), (II) and (IIc) in which A¹ is OH and A² is NH₂⁺ (or in which A¹ is NH₂ and A² is O) are referred to herein as rhodol dyes.

As highlighted by the above structures, when xanthene rings (or extended xanthene rings) are included in fluorescein, rhodamine and rhodol dyes, their carbon atoms are numbered differently. Specifically, their carbon atom numberings include primes. Although the above numbering systems for fluorescein, rhodamine and rhodol dyes are provided for convenience, it is to be understood that other numbering systems may be employed, and that they are not intended to be limiting. It is also to be understood that while one isomeric form of the dyes are illustrated, they may exist in other isomeric forms, including, by way of example and not limitation, other tautomeric forms or geometric forms. As a specific example, carboxy rhodamine and fluorescein dyes may exist in a lactone form.

In some embodiments, the fluorescent dye comprises a rhodamine dye. Exemplary suitable rhodamine dyes include, but are not limited to, rhodamine B, 5-carboxyrhodamine, rhodamine X (ROX), 4,7-dichlororhodamine X (dROX), rhodamine 6G (R6G), 4,7-dichlororhodamine 6G, rhodamine 110 (R110), 4,7-dichlororhodamine 110 (dR110), tetramethyl rhodamine (TAMRA) and 4,7-dichloro-tetramethylrhodamine (dTAMRA). Additional suitable rhodamine dyes include, for example, those described in U.S. Pat. Nos. 6,248,884, 6,111,116, 6,080,852, 6,051,719, 6,025,505, 6,017,712, 5,936,087, 5,847,162, 5,840,999, 5,750,409, 5,366,860, 5,231,191, and 5,227,487; PCT Publications WO 97/36960 and WO 99/27020; Lee et al., NUCL. ACIDS RES. 20:2471-2483 (1992), Arden-Jacob, NEUE LANWELLIGE XANTHEN-FARBSTOFFE FÜR FLUORESZENZSONDEN UND FARBSTOFF LASER, Verlag Shaker, Germany (1993), Sauer et al., J. FLUORESCENCE 5:247-261 (1995), Lee et al., NUCL. ACIDS RES. 25:2816-2822 (1997), and Rosenblum et al., NUCL. ACIDS RES. 25:4500-4504 (1997). A particularly preferred subset of rhodamine dyes are 4,7,-dichlororhodamines. In one embodiment, the fluorescent moiety comprises a 4,7-dichloro-orthocarboxyrhodamine dye.

In some embodiments, the fluorescent dye comprises a fluorescein dye. Exemplary suitable fluorescein include, but are not limited to, fluorescein dyes described in U.S. Pat. Nos. 6,008,379, 5,840,999, 5,750,409, 5,654,442, 5,188,934, 5,066,580, 4,933,471, 4,481,136 and 4,439,356; PCT Publication WO 99/16832, and EPO Publication 050684. A preferred subset of fluorescein dyes are 4,7-dichlorofluoresceins. Other preferred fluorescein dyes include, but are not limited to, 5-carboxyfluorescein (5-FAM) and 6-carboxyfluorescein (6-FAM). In one embodiment, the fluorescein moiety comprises a 4,7-dichloro-orthocarboxyfluorescein dye.

In some embodiments, the fluorescent dye can include a cyanine, a phthalocyanine, a squaraine, or a bodipy dye, such as those described in the following references and the references cited therein: U.S. Pat. Nos. 6,080,868, 6,005,113, 5,945,526, 5,863,753, 5,863,727, 5,800,996, and 5,436,134; and PCT Publication WO 96/04405.

In some embodiments, the fluorescent dye can comprise a network of dyes that operate cooperatively with one another such as, for example by FRET or another mechanism, to provide large Stoke's shifts. Such dye networks typically comprise a fluorescence donor moiety and a fluorescence acceptor moiety, and may comprise additional moieties that act as both fluorescence acceptors and donors. The fluorescence donor and acceptor moieties can comprise any of the previously described dyes, provided that dyes are selected that can act cooperatively with one another. In a specific embodiment, the fluorescent dye comprises a fluorescence donor dye which comprises a fluorescein dye and a fluorescence acceptor dye which comprises a fluorescein or rhodamine dye. Non-limiting examples of suitable dye pairs or networks are described in U.S. Pat. Nos. 6,399,392, 6,232,075, 5,863,727, and 5,800,996.

In some embodiments, the fluorescent moiety comprises a fluorescent lanthanide metal. Fluorescence properties of lanthanides are described in Lackowicz, 1999, Principles of Fluorescence Spectroscopy, 2nd Ed., Kluwar Academic, New York. Exemplary suitable lanthanide metals include, but are not limited to, europium (Eu³⁺) and terbium (Tb³⁺). In some embodiments, the fluorescent moiety comprises a chelated lanthanide. An exemplary chelate includes, but is not limited to, tetraisophthalmide (TIAM). In some embodiments, the fluorescent moiety comprises TIAM(Tb).

5.5 Charge-Moiety

The hydrophobic molecule can further comprise a charge-moiety, which when present, can discourage and/or inhibit micelle formation. The charge-moiety comprises any chemical group capable of carrying a charge. In some embodiments, the charge-moiety can be chemical group comprising an enzyme substrate. In some embodiments, the charge-moiety can be a chemical group comprising a dye moiety. In some embodiments, the charge-moiety can be chemical group used to link a dye moiety to the hydrophobic molecule.

In some embodiments, the charge-moiety comprises a net negative charge. In some embodiments, the charge-moiety comprises a net positive charge. Suitable examples of charge-moieties include dyes, amino acids, oligonucleotides and analogs and derivatives thereof.

In some embodiments, the charge-moiety comprises positively charged amino acids, such as arginine and lysine. Lysine and arginine contain side chains that carry a single positive charge at physiological pH. The imidazole side chain of histidine has a pKa of about 6, so it carries a full positive charge at a pH of about 6 or less. The charge-moiety can comprise negatively charged amino acids such as aspartic acid and glutamic acid. Aspartic acid and glutamic acid contain carboxyl side chains having a single negative charge. Cysteine has a pKa of about 8, so it carries a full negative charge at a pH above 8. The charge-moiety can comprise a phosphorylated amino acid. For example, a phosphoserine residue carries two negative charges on a phosphate group.

In some embodiments, the charge-moiety can further comprise uncharged amino acids, such as alanine, asparagine, cysteine, glutamine, glycine, isoleucine, leucine, methionine, phenylalanine, proline, tryptophan, and valine (i.e. physiological pH 6 to 9).

In some embodiments, the charge-moiety can comprise uncharged amino acids analogs. Suitable examples include 2-amino-4-fluorobenzoic acid, 2-amino-3-methoxybenzoic acid, 3,4-diaminobenzoic acid, 4-aminomethyl-L-phenylalanine, 4-bromo-L-phenylalanine, 4-cyano-L-proline, 3,4,-dihydroxy-L-phenylalanine, ethyl-L-tyrosine, 7-azaatryptophan, 4-aminohippuric acid, 2 amino-3-guanidinopropionic acid, L-citrulline, and derivatives.

In some embodiments, the charge-moiety can comprise positively charged amino acids analogs such as N-ω,ω-dimethyl-L-arginine, a-methyl-DL-ornithine, N-ω-nitro-L-arginine, and derivatives.

In some embodiments, the charge-moiety can comprise negatively charged amino acids analogs such as 2-aminoadipic acid, N-a-(4-aminobenzoyl)-L-glutamic acid, iminodiacetic acid, a-methyl-L-aspartic acid, a-methyl-DL-glutamic acid, y-methylene-DL-glutamic acid, and derivatives.

In some embodiments, the charge-moiety comprises an oligonucleotide. In some embodiments, the charge-moiety comprises deoxyribonucleotides (DNA). In some embodiments, the charge-moiety comprises ribonucleotides (RNA). In some embodiments, the charge-moiety comprises a combination of DNA and RNA.

In some embodiments, the charge-moiety comprises an oligonucleotide analog. The oligonucleotide analog can be a nucleobase polymers or oligomers in which the nucleobases are connected by a sugar phosphate backbone comprising one or more sugar phosphate analogs. Typical sugar phosphate analogs include, but are not limited to, sugar alkylphosphonates, sugar phosphoramidites, sugar alkyl- or substituted alkylphosphotriesters, sugar phosphorothioates, sugar phosphorodithioates, sugar phosphates and sugar phosphate analogs in which the sugar is other than 2′-deoxyribose or ribose, nucleobase polymers having positively charged sugar-guanidyl interlinkages.

In some embodiments, the charge-moiety comprises an oligonucleotide mimic. The oligonucleotide mimic can be a nucleobase polymer or oligomer in which one or more of the backbone sugar-phosphate linkages is replaced with a sugar-phosphate analog. In some embodiments, charge-moiety comprises a positively charged polyamide backbone, such as an alkylamine side chains. In some embodiments, charge-moiety comprises a negatively charged polyamide backbone. In some embodiments, the charge-moiety comprises an uncharged polyamide backbone. Non-limiting examples include, morpholino-phosphoramidate backbones, peptide-based nucleic acid mimic backbones, carbamate backbones, amide backbones, methylhydroxylamine backbones, 3′-thioformacetal backbones, and sulfamate backbones. In some embodiments, charge-moiety comprise a peptide nucleic acid (PNA) in which the nucleobases are connected by amino linkages.

In some embodiments, the charge-moiety comprises a peptide. In some embodiments, the peptide can comprise a substrate for an enzyme or agent. In some embodiments, the peptide comprises a length equal to or less than 30 amino acid residues, 25 residues, 20 residues, 15 residues, 10 residues, or 5 residues. In another embodiment, the peptide has a length in a range of 2 to 30 residues, or 2 to 25 residues, or 2 to 20 residues, or 2 to 15 residues, or 2 to 10 residues, or 2 to 5 residues, or 5 to 30 residues, or 5 to 25 residues, or 5 to 20 residues, or 5 to 15 residues, or 5 to 10 residues, or 10 to 30 residues, or 10 to 25 residues, or 10 to 20 residues, or 10 to 15 residues. In yet another embodiment, the peptide segment contains at least 2, 3, 4, 5, 6, 7, 8, 9, or 10 amino acid residues.

As described below, in some embodiments, the charge-moiety can comprise a substrate for an enzyme or agent.

5.6 Charge-Balance Molecule

The charge-balance molecule acts to promote or encourage micelle formation. Typically, the charge-balance molecule comprises sufficient opposite charge from the hydrophobic molecule to promote or encourage micelle formation. For example, if the hydrophobic molecule comprises one or more charged chemical groups (i.e. charge-moiety and dye moiety), the presence of these groups can destabilize the hydrophobic molecule in the micelle, thereby promoting the release of the hydrophobic molecule from the micelle in the absence of the specified enzyme. Release of the charged hydrophobic molecule from the micelle can be prevented or minimized by including a charge-balance molecule comprising sufficient opposite charge from the hydrophobic molecule so as to promote or encourage micelle formation. In some embodiments, the hydrophobic molecule can be negatively charged and the charge-balance molecule can be positively charged. In some embodiments, the hydrophobic molecule can be positively charged and charge-balance molecule can be negatively charged. Thus, by including a charge-balance molecule, micelles can be formed in the presence of destabilizing chemical groups in the hydrophobic molecule.

The charge-balance molecule can be designed to have a net negative or net positive charge by including an appropriate number of negatively and positively charged groups. For example, to establish a net positive charge (i.e., net charge ⁺2), the charge-balance molecule can be designed to contain positively charged groups, or a greater number of positively charged groups than negatively charged groups. To establish a net negative charge (i.e., net charge ⁻2), the charge-balance molecule can be designed to contain negatively charged groups, or a greater number of negatively charged groups than positively charged groups.

In designing a charge-balance molecule, the net charge depends in part, on a number of factors including the charge of the hydrophobic molecule. For example, in some embodiments, the hydrophobic molecule comprises a fluorescent dye and a charge-moiety, both of which can comprise one or more charged chemical groups that can destabilize or prevent micelle formation. By including a charge-balance molecule comprising sufficient opposite charge from the hydrophobic molecule micelle formation can be promoted or encouraged. Thus, the net charge of the charge-balance molecule, depends in part, on the presence of the charged groups comprising a hydrophobic molecule.

The overall charge of the charge-balance molecule also depends, in part upon other factors, such as, the molar ratio of the hydrophobic molecule to the charge-balance molecule, the pH of the assay medium, and concentration of salt in the assay medium.

The molar ratio of charge-balance molecule to hydrophobic molecule can be any ratio capable of promoting or encouraging micelle formation. In some embodiments, the molar ratio between the charge-balance molecule and hydrophobic molecule is about 1 to 1. In other embodiments, the molar ratio between the charge-balance molecule and hydrophobic molecule is about 9 to 1, 8 to 1, 7 to 1, 6 to 1, 5 to 1, 4 to 1, 3 to 1, 2 to 1. In other embodiments, the molar ratio between the charge-balance molecule and hydrophobic molecule is about 1 to 9, 1 to 8, 1 to 7, 1 to 6, 1 to 5, 1 to 4, 1 to 3, 1 to 2.

As a specific example, if the net charge of the hydrophobic molecule is ⁺2, an equal molar ratio of a charge-balance molecule with a net charge of ⁻2 can be used to promote or encourage micelle formation. In other embodiments, if the net charge of the hydrophobic molecule is ⁺2, a charge-balance molecule with a net charge of ⁻1 can be used to promote or encourage micelle formation at a 1:2 molar ratio of hydrophobic molecule to charge-balance molecule. As another specific example, if the net charge of the hydrophobic molecule is ⁻5, a non-equal molar ratio of a charge-balance molecule with a net charge of ⁺18 can be used to promote or encourage micelle formation.

Another factor affecting the charge of the charge-balance molecule is the pH of the assay medium and the pKas' of the groups comprising the charge-balance molecule. For example, in some embodiments, if the charge-balance molecule is designed to carry a positive charge at pH 7.6, then amino acids with side chains having pKas' above 7.6 can be chosen i.e. lysine (pKa 10.5) and arginine (pKa 12.5) carry a positive charge at pH 7.6. In some embodiments, if the charge-balance molecule is designed to carry a negative charge at pH 7.6, then amino acids with side chains having pKas' below 7.6 can be chosen i.e. aspartic acid (pKa 3.9) and glutamic acid (pKa 4.3) carry a negative charge at pH 7.6. The pKa values of the common amino acids at different pHs are shown in

TABLE 3
Table 3¹

| Amino Acid (IUPAC) | α-COOH pKa | α-NH₃⁺ pKa | Side chain pKa |
|---|---|---|---|
| Alanine (A) | 2.4 | 9.7 | |
| Cysteine (C) | 1.7 | 10.8 | 8.3 |
| Aspartic acid (D) | 2.1 | 9.8 | 3.9 |
| Glutamic acid (E) | 2.2 | 9.7 | 4.3 |
| Phenylalanine (F) | 1.8 | 9.1 | |
| Glycine (G) | 2.3 | 9.6 | |
| Histidine (H) | 1.8 | 9.2 | 6.0 |
| Isoleucine (I) | 2.4 | 9.7 | |
| Lysine (K) | 2.2 | 9.0 | 10.5 |
| Leucine (L) | 2.4 | 9.6 | |
| Methionine (M) | 2.3 | 9.2 | |
| Asparagine (N) | 2.0 | 8.8 | |
| Proline (P) | 2.1 | 10.6 | |
| Glutamine (Q) | 2.2 | 9.1 | |
| Arginine (R) | 2.2 | 9.0 | 12.5 |
| Serine (S) | 2.2 | 9.2 | ˜13 |
| Threonine (T) | 2.6 | 10.4 | ˜13 |
| Valine (V) | 2.3 | 9.6 | |
| Tryptophan (W) | 2.4 | 9.4 | |
| Tyrosine Y | 2.2 | 9.1 | 10.1 |

¹Garerett, R. H. and Grisham M. Biochemistry 2nd edition (1999) Saunders College Publishing. The pKa values depend on temperature, ionic strength, and the microenvironment of the ionizable group.

The charge-balance molecule comprises any group capable of carrying a charge. Non-limiting examples of groups include metal ions, primary amines, secondary amines, tertiary amines, ammonium groups, metal ions, amino acids, peptides, proteins, oligonucleotides and combinations thereof.

In some embodiments, the charge-balance molecule comprises a metal ion. Non-limiting examples of metal ions that can be used include magnesium, manganese, lanthanum and any combination thereof.

In some embodiments, the charge-balance molecule comprises an oligonucleotide. In some embodiments, the charge-balance molecule comprises deoxyribonucleotides (DNA). In some embodiments, the charge-balance molecule comprises ribonucleotides (RNA). In some embodiments, the charge-balance molecule comprises a combination of DNA and RNA.

In some embodiments, the charge-balance molecule comprises an oligonucleotide analog. The oligonucleotide analog can be a nucleobase polymers or oligomers in which the nucleobases are connected by a sugar phosphate backbone comprising one or more sugar phosphate analogs. Typical sugar phosphate analogs include, but are not limited to, sugar alkylphosphonates, sugar phosphoramidites, sugar alkyl- or substituted alkylphosphotriesters, sugar phosphorothioates, sugar phosphorodithioates, sugar phosphates and sugar phosphate analogs in which the sugar is other than 2′-deoxyribose or ribose, nucleobase polymers having positively charged sugar-guanidyl interlinkages.

In some embodiments, the charge-balance molecule comprises an oligonucleotide mimic. The oligonucleotide mimic can be a nucleobase polymer or oligomer in which one or more of the backbone sugar-phosphate linkages is replaced with a sugar-phosphate analog. In some embodiments, charge-balance molecule comprises a positively charged polyamide backbone, such as an alkylamine side chains. In some embodiments, charge-balance molecule comprises a negatively charged polyamide backbone. In some embodiments, the charge-balance molecule comprises an uncharged polyamide backbone. Non-limiting examples include, morpholino-phosphoramidate backbones, peptide-based nucleic acid mimic backbones, carbamate backbones, amide backbones, methylhydroxyl amine backbones, 3′-thioformacetal backbones, and sulfamate backbones. In some embodiments, the charge-balance molecule comprise a peptide nucleic acid (PNA) in which the nucleobases are connected by amino linkages.

In some embodiments the charge-balance molecule comprises a charged amino acid or amino acid analogs. In some embodiments, the charge-balance comprises positively charged amino acids such as arginine and lysine. In some embodiments, the charge-balance molecule can comprise positively charged amino acids analogs such as N-ω,ω-dimethyl-L-arginine, a-methyl-DL-ornithine, N-ω-nitro-L-arginine, and derivatives.

In some embodiments, the charge-balance molecule comprises negatively charged amino acids such as aspartic acid and glutamic acid. Aspartic acid and glutamic acid contain carboxyl side chains having a single negative charge. Cysteine has a pKa of about 8, so it carries a full negative charge at a pH above 8. In some embodiments, the charge-balance molecule comprises a phosphorylated amino acid or analog. For example, a phosphoserine residue carries two negative charges on a phosphate group. In some embodiments, the charged moiety can comprise negatively charged amino acids analogs such as 2-aminoadipic acid, N-a-(4-aminobenzoyl)-L-glutamic acid, iminodiacetic acid, a-methyl-L-aspartic acid, a-methyl-DL-glutamic acid, y-methylene-DL-glutamic acid, and derivatives.

In some embodiments, the charge-balance molecule can further comprise uncharged amino acids such as alanine, asparagine, cysteine, glutamine, glycine, isoleucine, leucine, methionine, phenylalanine, proline, tryptophan, and valine. In some embodiments, charge-balance molecule comprises uncharged amino acids analogs. Suitable examples include 2-amino-4-fluorobenzoic acid, 2-amino-3-methoxybenzoic acid, 3,4-diaminobenzoic acid, 4-aminomethyl-L-phenylalanine, 4-bromo-L-phenylalanine, 4-cyano-L-proline, 3,4,-dihydroxy-L-phenylalanine, ethyl-L-tyrosine, 7-azaatryptophan, 4-aminohippuric acid, 2 amino-3-guanidinopropionic acid, L-citrulline, and derivatives.

In some embodiments, the charge-balance molecule can comprise a peptide. In some embodiments, the peptide can comprise a substrate for an enzyme or agent. In some embodiments, the peptide comprises a length equal to or less than 30 amino acid residues, 25 residues, 20 residues, 15 residues, 10 residues, or 5 residues. In another embodiment, the peptide has a length in a range of 2 to 30 residues, or 2 to 25 residues, or 2 to 20 residues, or 2 to 15 residues, or 2 to 10 residues, or 2 to 5 residues, or 5 to 30 residues, or 5 to 25 residues, or 5 to 20 residues, or 5 to 15 residues, or 5 to 10 residues, or 10 to 30 residues, or 10 to 25 residues, or 10 to 20 residues, or 10 to 15 residues. In yet another embodiment, the peptide segment contains at least 2, 3, 4, 5, 6, 7, 8, 9, or 10 amino acid residues. In some embodiments, charge-balance molecule can comprise the peptide E-E-I-Y-G-E-F (SEQ ID NO:1). In some embodiments, charge-balance molecule can comprise the peptide K-K-A-A-G-K-L (SEQ ID NO: 2).

In some embodiments, the charge-balance molecule comprises a charged protein. In these embodiments, the concentration of the charged protein is about 2, 3, 4, 5, 6, 7, 8, 9, or 10-times greater than the concentration of endogenous charged protein in a sample. In some embodiments, the charge-balance molecule charged protein and the endogenous charged protein in the sample are the same protein. In some embodiments, the charge-balance molecule charged protein and the endogenous charged protein in the sample are different proteins. Non-limiting examples of charged proteins that can be used include myelin basic protein (MBP), myelin P2 protein, and casein.

In some embodiments, the micelle can comprise more than one charge-balance molecule. Any combination of charge-balance molecules capable of promoting or encouraging micelle formation can be used. In some embodiments, the micelle can comprise charge-balance molecules comprising the same group capable of carrying a charge. In some embodiments, the micelle can comprise charge-balance molecules comprising different groups capable of carrying a charge. For example, in a specific embodiment, the micelle can comprise a charge-balance molecule comprising a metal ion and charge-balance molecule comprising a protein.

5.7 Substrate

The hydrophobic molecule and/or charge-balance molecule comprise a substrate or putative substrate that can be acted upon by enzymes or agents. In some embodiments, the optional charge-moiety comprises an enzyme substrate. In some embodiments, the hydrophobic molecule and/or charge-balance molecule, can each independently of the other, comprise a substrate or putative substrate for enzymes or agents of interest. In some embodiments, the hydrophobic molecule and the charge-balance molecule both comprise the same substrate.

In some embodiments, the hydrophobic molecule comprises one substrate. In some embodiments, the hydrophobic molecule comprises two, three, four, or more substrates, wherein the substrates can be the same or different. The substrates can be connected in any way that permits them to perform their respective function. In some embodiments, the substrates can be directly connected to each other. In other embodiments, the substrates can be indirectly connected to each other via one or more linkage groups. In yet other embodiments, the substrates can be indirectly linked to each other through a dye moiety or a hydrophobic moiety.

In some embodiments, the charge-balance molecule comprises one substrate. In some embodiments, the charge-balance molecule comprises two, three, four, or more substrates, wherein the substrates can be the same or different. The substrates can be connected in any way that permits them to perform their respective function. In some embodiments, the substrates can be directly connected to each other. In other embodiments, the substrates can be indirectly connected to each other via one or more linkage groups. In yet other embodiments, the substrates can be indirectly linked to each other through a dye moiety.

In some embodiments, the charge-balance molecule comprises one substrate. In some embodiments, the charge-balance molecule comprises two, three, four, or more substrates, wherein the substrates can be the same or different. The substrates can be connected in any way that permits them to perform their respective function. In some embodiments, the substrates can be directly connected to each other. In other embodiments, the substrates can be indirectly connected to each other via one or more linkage groups. In yet other embodiments, the substrates can be indirectly linked to each other through a dye moiety.

A substrate can comprise a substrate or putative substrate that can be acted upon by specified enzymes or agents. Any type of enzyme or chemical reactions on the substrate/micelle may be used, provided that it is capable of producing a detectable change (e.g., an increase) in fluorescence. Preferably, the specified enzyme is substantially active at the interface between the micelle and the assay medium. Selection of a particular enzyme or chemical reaction on the substrate, can depend, in part, on the structure of the hydrophobic molecule and/charge balance molecule, as well as on other factors.

In some embodiments, the enzymes or agents act upon the substrate to cleave the substrate. In these embodiments, the substrate comprises a cleavage site that is cleavable by a chemical reagent or cleaving enzyme. As a specific example, the substrate can comprise a cleavage site that is cleavable by a lipase, a phospholipase, a peptidase, a nuclease or a glycosidase enzyme. The substrate may further comprise additional residues and/or features that facilitate the specificity, affinity and/or kinetics of the cleaving enzyme. Depending upon the requirements of the particular cleaving enzyme, such cleaving enzyme “recognition moieties” can comprise the cleavage site or, alternatively, the cleavage site may be external to the recognition moiety. For example, certain endonucleases cleave at positions that are upstream or downstream of the region of the nucleic acid molecule bound by the endonuclease.

The chemical composition of the substrate will depend upon, among other factors, the requirements of the cleaving enzyme. For example, if the cleaving enzyme is a protease, the substrate can comprise a peptide (or analog thereof) recognized and cleaved by the particular protease. If the cleaving enzyme is a nuclease, the substrate can comprise an oligonucleotide (or analog thereof) recognized and cleaved by a particular nuclease. If the cleaving enzyme is a phospholipase, the substrate moiety can comprise a diacylglycerolphosphate group recognized and cleaved by a particular phospholipase.

Sequences and structures recognized and cleaved by the various different types of cleaving enzymes are well known. Any of these sequences and structures can comprise the substrate. Although the cleavage can be sequence specific, in some embodiments it can be non-specific. For example, the cleavage can be achieved through the use of a non-sequence specific nuclease, such as, for example, an RNase.

Cleavage of the substrate by the corresponding cleaving enzyme can release the fluorescent dye from the micelle, reducing or eliminating its quenching and producing a measurable increase in fluorescence.

In other embodiments, the enzymes or agents act upon the substrate by the addition, deletion, or substitution of chemical moieties to the substrate. These reactions can destabilize the hydrophobic molecule and/or charge-balance molecule in the micelle, thereby promoting its release from the micelle.

As a specific example, in some embodiments, the enzymes or agents act upon the substrate to change the net charge of the substrate, such as by phosphorylation of one or more unphosphorylated residues by a kinase enzyme or dephosphorylation of one or more phosphorylated residues by a phosphatase enzyme. Specific examples of substrates modifiable by protein kinase and phosphatase enzymes are described in more detail below.

By way of illustration, the substrate is first discussed below with reference to protein kinases as exemplary enzymes to be detected, quantified, and/or characterized. In addition to playing important biochemical roles, protein kinases are also useful for illustrating enzymes that cause an increase in the net charge of a substrate by adding a phosphate group to a hydroxyl group to form a phosphorylated substrate. Under physiological conditions, i.e. pH 6 to pH 9, phosphorylation of the substrate causes the addition of two negative charges, for a net change in charge of ⁻2. Enzymes that carry out the opposite reaction, protein phosphatases, are also discussed, which cause a net increase in charge of ⁺2 in the substrate, under physiological conditions, i.e. pH 6 to pH 9. In either case, the amplitude of the net charge on the substrate is increased. For example, upon phosphorylation of a substrate as described above, the amplitude of the net negative charge on the substrate is increased by ⁻2. On the other hand, upon dephosphorylation of a substrate by a phosphatase, the amplitude of the net positive charge on the substrate is increased by ⁺2.

In some embodiments, a substrate for detecting, quantifying and/or characterizing one or more protein kinases in a sample is provided. The protein kinase substrate generally comprises an amino acid side chain containing a group that is capable of being phosphorylated by a protein kinase. In some embodiments, the phosphorylatable group is a hydroxyl group. Usually, the hydroxyl group is provided as part of a side chain in a tyrosine, serine, or threonine residue, although any other natural or non-natural amino acid side chain or other entity containing a phosphorylatable hydroxyl group can be used. The phosphorylatable group can also be a nitrogen atom, such as the nitrogen atom in the epsilon amino group of lysine, an imidazole nitrogen atom of histidine, or a guanidinium nitrogen atom of arginine. The phosphorylatable group can also be a carboxyl group in an asparate or glutamate residue.

The protein kinase substrate can further comprise a segment, typically a polypeptide segment, that contains one or more subunits or residues (in addition to the phosphorylatable residue) that impart identifying features to the substrate to make it compatible with the substrate specificity of the protein kinase(s) to be used to be detected, quantified, and/or characterized.

A wide variety of protein kinases have been characterized over the past several decades, and numerous classes have been identified (see, e.g., S. K. Hanks et al., Science 241:42-52 (1988); B. E. Kemp and R. B. Pearson, Trends Biochem. Sci. 15:342-346 (1990); S. S. Taylor et al., Ann. Rev. Cell Biol. 8:429-462 (1992); Z. Songyang et al., Current Biology 4:973-982 (1994); and Chem. Rev. 101:2209-2600, “Protein Phosphorylation and Signaling” (2001)). Exemplary classes of protein kinases include cAMP-dependent protein kinases (also called the protein kinase A family, A-proteins, or PKA's), cGMP-dependent protein kinases, protein kinase C enzymes (PKC's, including calcium dependent PKC's activated by diacylglycerol), Ca²⁺/calmodulin-dependent protein kinase I or II, protein tyrosine kinases (e.g., PDGF receptor, EGF receptor, and Src), mitogen activated protein (MAP) kinases (e.g., ERK1, KSS1, and MAP kinase type I), cyclin-dependent kinases (CDk's, e.g., Cdk2 and Cdc2), and receptor serine kinases (e.g., TGF-β). Exemplary consensus sequences and/or enzyme substrates for various protein kinases are shown in Table 4, below. As will be appreciated by a person skilled in the art, these various consensus sequences and enzyme substrates can be used to design protein kinase recognition moieties having desired specificities for particular kinases and/or kinase families.

TABLE 4

| | | Consensus Sequence^{a}/ |
|---|---|---|
| Symbol | Description | Enzyme Substrates |
| PKA | cAMP-dependent | -R-R-X-S/T-Z- (SEQ ID NO:3) |
| | | -L-R-R-A-S-L-G- (SEQ ID NO:4) |
| PhK | phosphorylase kinase | -R-X-X-S/T-F-F- (SEQ ID NO:5) |
| | | -R-Q-G-S-F-R-A- (SEQ ID NO:6) |
| cdk2 | cyclin-dependent | -S/T-P-X-R/K (SEQ ID NO:7) |
| | kinase-2 | |
| ERK2 | extracellular- | -P-X-S/T-P (SEQ ID NO:8) |
| | regulated kinase-2 | -R-R-I-P-L-S-P (SEQ ID NO:7) |
| PKC | protein kinase C | K-K-K-K-R-F-S-F-K^{b} (SEQ ID NO:9) |
| | | X-R-X-X-S-X-R-X (SEQ ID NO:10) |
| CaMKI | Ca²⁺/calmodulin- | L-R-R-L-S-D-S-N-F^{c} (SEQ ID NO:11) |
| | dependent protein | |
| | kinase I | |
| CaMKII | Ca²⁺/calmodulin- | K-K-L-N-R-T-L-T-V-A^{d} (SEQ ID NO:12) |
| | dependent protein | |
| | kinase II | |
| c-Src | cellular form of Rous | -E-E-I-Y-E/G-X-F (SEQ ID NO:13) |
| | sarcoma virus trans- | -E-E-I-Y-G-E-F-R (SEQ ID NO:14) |
| | forming agent | |
| v-Fps | transforming agent of | -E-I-Y-E-X-I/V (SEQ ID NO:15) |
| | Fujinami sarcoma virus | |
| Csk | C-terminal Src kinase | -I-Y-M-F-F-F (SEQ ID NO: 16) |
| InRK | Insulin receptor | -Y-M-M-M (SEQ ID NO:17) |
| | kinase | |
| EGFR | EGF receptor | -E-E-E-Y-F (SEQ ID NO:18) |
| SRC | Src kinase | -R-I-G-E-G-T-Y-G-V-V-R-R- |
| | | (SEQ ID NO:19) |
| Akt | RAC-beta serine/ | -R-P-R-T-S-S-F- (SEQ ID NO:20) |
| | threonine-protein | |
| | kinase | |
| Erk1 | Extracellular signal- | -P-R-T-P-G-G-R- (SEQ ID NO:21) |
| | regulated kinase 1 | |
| | (MAP kinase 1, MAPK 1) | |
| MAPKAPK2 | MAP kinase-activated | -R-L-N-R-T-L-S-V (SEQ ID NO:22) |
| | protein kinase 2 | |
| NEK2 | Serine/threonine- | -D-R-R-L-S-S-L-R (SEQ ID NO:23) |
| | protein kinase Nek2 | |
| Ab1 | tyrosine kinase | -E-A-I-Y-A-A-P-F-A-R-R-R |
| | | (SEQ ID NO:24) |
| YES | Proto-oncogene tyro- | E-E-I-Y-G-E-F-R (SEQ ID NO:25) |
| | sine-protein kinase | |
| | YES | |
| LCK | Proto-oncogene tyro- | E-E-I-Y-G-E-F-R (SEQ ID NO:25) |
| | sine-protein kinase | |
| | LCK | |
| SRC | Proto-oncogene tyro- | K-V-E-K-I-G-E-G-T-Y-G-V-V-Y-K |
| | sine-protein kinase | (SEQ ID NO:26) |
| | Src | |
| LYN | Tyrosine-protein | E-E-E-I-Y-G-E-F (SEQ ID NO:26) |
| | kinase LYN | |
| BTK | Tyrosine-protein | E-E-I-Y-G-E-F-R- (SEQ ID NO:27) |
| | kinase BTK | |
| GSK3 | Glycogen synthase | R-H-S-S-P-H-Q-(Sp)-E-D-E-E |
| | kinase-3 | (SEQ ID NO:28) |
| CKI | Casein kinase I | R-R-K-D-L-H-D-D-E-E-D-E-A- |
| | | M-S-I-T-A (SEQ ID NO:29) |
| CKII | Casein kinase II | -(Sp)-X-X-S/T- (SEQ ID NO:30) |
| | | S-X-X-E/D (SEQ ID NO:31) |
| | | R-R-R-D-D-D-S-D-D-D (SEQ ID NO:30) |
| TK | Tyrosine kinase | K-G-P-W-L-E-E-E-E-E-A-Y-G- |
| | | W-L-D-F (SEQ ID NO:32) |

^{a}see, for example, B.E. Kemp and R.B. Pearson, Trends Biochem. Sci. 15:342-346 (1990); Z. Songyang et al., Current Biology 4:973-982 (1994); J.A. Adams, Chem Rev. 101:2272 (2001) and references cited therein; X means any amino acid residue, “/” indicates alternate residues; and Z is a hydrophobic amino acid, such as valine, leucine or isoleucine
^{b}Graff et al., J. Biol. Chem. 266:14390-14398 (1991)
^{c}Lee et al., Proc. Natl. Acad. Sci. 91:6413-6417 (1994)
^{d}Stokoe et al., Biochem. J. 296:843-849 (1993).

Protein kinase substrates having desired specificities for particular kinases and/or kinase families can also be designed, for example, using the methods and/or exemplary sequences described in Brinkworth et al., Proc. Natl. Acad. Sci. USA 100(1):74-79 (2003).

Typically, the protein kinase substrates comprise a sequence of L-amino acid residues. However, any of a variety of amino acids with different backbone or sidechain structures can also be used, such as: D-amino acid polypeptides, alkyl backbone moieties joined by thioethers or sulfonyl groups, hydroxy acid esters (equivalent to replacing amide linkages with ester linkages), replacing the alpha carbon with nitrogen to form an aza analog, alkyl backbone moieties joined by carbamate groups, polyethyleneimines (PEIs), and amino aldehydes, which result in polymers composed of secondary amines. A more detailed backbone list includes N-substituted amide (—CON(R)— replaces —CONH— linkages), esters (—CO₂—), keto-methylene (—COCH₂—)methyleneamino (—CH₂NH—), thioamide (—CSNH—), phosphinate (—PO₂RCH₂—), phosphonamidate and phosphonamidate ester (—PO₂RNH₂), retropeptide (—NHC(O)—), trans-alkene (—CR═CH—), fluoroalkene (e.g.; —CF═CH—), dimethylene (—CH₂CH₂—), thioether (e.g.; —CH₂SCH₂—), hydroxyethylene (—CH(OH)CH₂—), methyleneoxy (—CH₂O—), tetrazole (—CN₄—), retrothioamide (—NHC(S)—), retroreduced (—NHCH₂—), sulfonamido (—SO₂NH—), methylenesulfonamido (—CHRSO₂NH—), retrosulfonamide (—NHS(O₂)—), and peptoids (N-substituted glycines), and backbones with malonate and/or gem-diaminoalkyl subunits, for example, as reviewed by M. D. Fletcher et al., Chem. Rev. 98:763 (1998) and the references cited therein. Peptoid backbones (N-substituted glycines) can also be used (e.g., H. Kessler, Angew. Chem. Int. Ed. Engl. 32:543 (1993); R. N. Zuckermann, Chemtracts-Macromol. Chem. 4:80 (1993); and Simon et al., Proc. Natl. Acad. Sci. 89:9367 (1992)).

In some embodiments, the protein kinase substrate includes all of the residues comprising the recognition sequence for a given protein kinase. The total number of residues comprising the recognition sequence can be defined as N, wherein N is an integer from 1 to 10. In some embodiments, N is an integer from 1 to 15. In other embodiments, N is an integer from 1 to 20. As a specific example of these embodiments, the consensus recognition sequence for PKA is -R-R-X-S/T-Z, thus, N=5. Repetition of the recognition sequence, two, three, or four, or more times can be used to provide a protein kinase substrate comprising two, three, four or more unphosphorylated residues.

In other embodiments, the protein kinase substrate comprises overlapping recognition sequences. In these embodiments, one or more residues from a recognition sequence are shared between two recognition sequences. As a specific example of these embodiments, the consensus recognition sequence for p38βII is P-X-S-P. A recognition moiety with overlapping consensus sequences can be created by sharing a -P- residue between two recognition sequences, e.g., P-X-S-P-X-S-P.

In other embodiments, the protein kinase substrate can comprise a subset of the residues comprising the recognition sequence. In these embodiments, one or more residues are omitted from the recognition motif. A subset is defined herein as comprising N-u amino acid residues, wherein, as defined above, N represents the total number of amino acid residues comprising the recognition sequence, and u represents the number of amino acid residues omitted from the recognition sequence. In some embodiments, u is an integer from 1 to 9. In other embodiments, u is an integer from 1 to 14. In still other embodiments, u is an integer from 1 to 19. For example, if the total number of amino acids in the recognition motif is 4, subsets comprising 3, 2, or 1 amino acid residue(s) can be made. If the total number of amino acids in the recognition motif is 5, subsets comprising 4, 3, 2, or 1 amino acid residue(s) can be made. If the total number of amino acids in the recognition motif is 6, subsets comprising 5, 3, 2, or 1 amino acid residue(s) can be made. If the total number of amino acids in the recognition motif is 7, subsets comprising 6, 5, 4, 3, 2, or 1 amino acids residue(s) can be made. If the recognition motif comprises 8 amino acids, subsets comprising 7, 6, 5, 4, 3, 2, or 1 amino acid residue(s) can be made. If the total number of amino acids in the recognition motif is 9, subsets comprising 8, 7, 6, 5, 4, 3, 2, or 1 amino acids residue(s) can be made. If the recognition motif comprises 10 amino acids, subsets comprising 9, 8, 7, 6, 5, 4, 3, 2, or 1 amino acids residue(s) can be made. Typically, subsets comprising N−1 or N-2 amino acid residues are made.

The number of residues to include in the recognition sequence, in part, will depend, on the specificity of the protein kinase. For example, some protein kinases, such as p38βII, require all of the residues comprising the recognition sequence to be present for phosphorylation activity to occur. Other protein kinases, such as PKC, can phosphorylate a recognition sequence, in which one or more residues are omitted from the recognition sequence. In other embodiments, recognition sequences comprising a unphosphorylated residue are designed for use with protein kinases, such as GSK3, that require a phosphorylated residue in order to phosphorylate one or more unphophosphorylated residue.

Various combinations of the foregoing embodiments can be used in the compositions and methods described herein. For example, kinase substrates comprising recognition moieties that include recognition sequences comprising N residues for a given protein kinase can be selected. In other embodiments, kinase substrates comprising recognition moieties, in which one recognition sequence comprises N residues and the other recognition sequence comprises N-u residues can be selected. Thus, substrate compounds comprising recognition moieties with any combination of N and N-u recognition sequences can be used, provided there is a detectable increase in fluorescence when the protein kinase is present. Moreover, the recognition moieties can be for the same protein kinase, or they may be for different protein kinases.

The distance between unphosphorylated residues depends, in part, on the location of the unphosphorylated residue(s) in each of the selected recognition sequences, and, in part, on the way in which the selected recognition sequences are connected. Unphosphorylated residues capable of being phosphorylated by a protein kinase can be adjacent, or they can be separated by one, two, three, or more residues that are not phosphorylated by a protein kinase. For example, a substrate compound in which the unphosphorylated residues are separated by three residues can be formed by connecting two recognition sequences, each comprising the recognition sequence -S-X-X-X- to each other to form a recognition moiety having the composition -S-X-X-X-S-X-X-X-. In another example, a substrate compound, in which the unphosphorylated residues are separated by two residues can be formed by sharing an amino acid residue between two recognition sequences, e.g., the -P- in the recognition sequence -P-X-S-P-can be shared to form the recognition moiety -P-X-S-P-X-S-P-. Thus, any combination of N and N-u recognition sequences, in which the unphosphorylated residues are adjacent, or are separated by one or more residues, can be used in the kinase substrates provided that an increase in fluorescence is observed in the presence of the protein kinase(s).

The protein kinase recognition sequences can be connected in any way that permits them to perform their respective function. In some embodiments, the protein kinase recognition sequences can be directly connected to each other. In other embodiments, the protein kinase recognition sequences can be indirectly connected to each other via one or more linkage groups. In yet other embodiments, the protein kinase recognition moieties can be indirectly linked to each other through the fluorescent moiety or the hydrophobic moiety.

In some embodiments, the charge-balance molecule comprises a kinase substrate. In some embodiments, the kinase substrate can be a whole protein, for example, a myelin basic protein. In some embodiments, myelin basic protein is acted on by a kinase selected from PKA, PKC, MAPK, calmodulin-dependent protein kinase, phosphorylase kinase, Rafl, MEK, MEKK and any combination thereof.

In another aspect, a substrate for detecting, quantifying, and/or characterizing one or more protein phosphates in a sample is provided. Also, the phosphatase can be a phosphatase candidate, and the methods used to confirm and/or characterize the phosphatase activity of the candidate.

A wide variety of protein phosphatases have been identified (e.g., see P. Cohen, ANN. REV. BIOCHEM. 58:453-508 (1989); MOLECULAR BIOLOGY OF THE CELL, 3rd edition Alberts et al., eds., Garland Publishing, NY (1994); and CHEM. REv. 101:2209-2600, “Protein Phosphorylation and Signaling” (2001)). Serine/threonine protein phosphatases represent a large class of enzymes that reverse the action of protein kinases, such as PKAs. The serine/threonine protein phosphatases have been divided among four groups designated I, IIA, IIB, and IIC. Protein tyrosine kinases are also an important class of phosphatase. Histidine, lysine, arginine, and asparate phosphatases are also known (e.g., P. J. Kennelly, CHEM REV. 101:2304-2305 (2001) and references cited therein). In some cases, phosphatases are highly specific for only one or a few proteins, but in other cases, phosphatases are relatively non-specific and can act on a large range of protein targets. Examples of peptide sequences that can be dephosphorylated by phosphatases are described in P. J. Kennelly, supra.

The substrate can be designed to be reactive with a particular phosphatase or a group of phosphatases. The unphosphorylated residue in the phosphatase recognition sequence may be any group that is capable of being dephosphorylated by a phosphatase. In some embodiments, the residue is a phosphotyrosine residue. In some embodiments, the residue is a phosphoserine residue. In some embodiments, the residue is a phosphothreonine residue.

The phosphatase recognition moiety may further comprises a segment, typically a polypeptide segment, that contains one or more subunits or residues (in addition to the dephosphorylatable residues) that impact identifying features to the recognition site to make it compatible with the substrate specificity of the protein phosphatase(s) to be used to modify the signal molecule.

The protein kinase or phosphatase recognition moiety may comprise a polypeptide segment containing the group or residue that is to be phosphorylated or dephosphorylated. In some embodiments, such a polypeptide segment has a polypeptide length equal to or less than 30 amino acid residues, 25 residues, 20 residues, 15 residues, 10 residues, or 5 residues. In another embodiment, the polypeptide segment has a polypeptide length in a range of 3 to 30 residues, or 3 to 25 residues, or 3 to 20 residues, or 3 to 15 residues, or 3 to 10 residues, or 3 to 5 residues, or 5 to 30 residues, or 5 to 25 residues, or 5 to 20 residues, or 5 to 15 residues, or 5 to 10 residues, or 10 to 30 residues, or 10 to 25 residues, or 10 to 20 residues, or 10 to 15 residues. In yet another embodiment, the polypeptide segment contains at least 3, 4, 5, 6 or 7 amino acid residues.

In addition to having one or more phosphorylated residues capable of being dephosphorylated, the phosphatase substrate can include additional amino acid residues (or analogs thereof) that facilitate binding specificity, affinity, and/or rate of dephosphorylation by the phosphatase.

Phosphatase substrates having desired specificities for particular phosphatase and/or phosphatase families can be designed as described above for exemplary protein kinase consensus sequences, provided that at least one residue is phosphorylated. The phosphatase to be detected or characterized can be any phosphatase known in the art. In some embodiments, the phosphate can be a phosphatase 2C, an alkaline phosphatase, or a tyrosine phosphatase.

In some embodiments, a substrate for detecting or characterizing one or more sulfatases in a sample is provided. A wide variety of sulfatases have been identified. In some cases, sulfatases are highly specific for only one or a few substrates, but in other cases, sulfatases are relatively non-specific and can act on a large range of substrates including, but not limited to, proteins, glycosaminoglycans, sulfolipids, and steroid sulfates. Exemplary sulfatases and sulfatase substrates are shown in Table 5, below. These substrates can be used to design sulfatase recognition moieties having desired specificities for particular sulfatases and/or sulfatase families.

TABLE 5

| Sulfatase Description | EC | |
|---|---|---|
| (Alternative Name(s)) | number | Substrate(s) |
| Arylsulfatase | 3.1.6.1 | phenol sulfate |
| (Sulfatase; Aryl-sulphate, | | |
| sulphohydrolase) | | |
| Steryl-sulfatase | 3.1.6.2 | 3-beta-hydroxyandrost-5-en-17- |
| (Steroid sulfatase; Steryl- | | one 3-sulfate and related steryl |
| sulfate sulfohydrolase; | | sulfates |
| Arylsulfatase C) | | |
| Glucosulfatase | 3.1.6.3 | D-glucose 6-sulfate and other |
| | | sulfates of mono- and |
| | | disaccharides and on adenosine |
| | | 5′-sulfate |
| N-acetylgalactosamine-6- | 3.1.6.4 | 6-sulfate groups of the N- |
| sulfatase | | acetyl-D-galactosamine; 6- |
| (Chondroitinsulfatase, | | sulfate units of chondroitin |
| Chondroitinase, Galactose-6- | | sulfate and of the D-galactose |
| sulfate sulfatase) | | 6-sulfate units of keratan |
| | | sulfate. |
| Choline-sulfatase | 3.1.6.6 | Choline sulfate |
| Cellulose-polysulfatase | 3.1.6.7 | 2- and 3-sulfate groups of the |
| | | polysulfates of cellulose and |
| | | charonin |
| Cerebroside-sulfatase | 3.1.6.8 | A cerebroside 3-sulfate; |
| (Arylsulfatase A) | | galactose 3-sulfate residues in a |
| | | number of lipids; ascorbate 2- |
| | | sulfate; phenol sulfates |
| Chondro-4-sulfatase | 3.1.6.9 | 4-deoxy-beta-D-gluc-4- |
| | | enuronosyl-(1,4)-N-acetyl-D- |
| | | galactosamine 4-sulfate |
| Chondro-6-sulfatase | 3.1.6.10 | 4-deoxy-beta-D-gluc-4- |
| | | enuronosyl-(1,4)-N-acetyl-D- |
| | | galactosamine 6-sulfate; N- |
| | | acetyl-D-galactosamine 4,6- |
| | | disulfate |
| Disulfoglucosamine-6- | 3.1.6.11 | N,6-O-disulfo-D-glucosamine |
| sulfatase | | |
| (N-sulfoglucosamine-6- | | |
| sulfatase) | | |
| N-acetylgalactosamine-4- | 3.1.6.12 | 4-sulfate groups of the N- |
| sulfatase | | acetyl-D-galactosamine; 4- |
| (Arylsulfatase B; | | sulfate units of chondroitin |
| Chondroitinsulfatase; | | sulfate; dermatan sulfate; N- |
| Chondroitinase) | | acetylglucosamine 4-sulfate |
| Iduronate-2-sulfatase | 3.1.6.13 | 2-sulfate groups of the L- |
| (Chondroitinsulfatase) | | iduronate; 2-sulfate units of |
| | | dermatan sulfate; heparan |
| | | sulfate and heparin. |
| N-acetylglucosamine-6- | 3.1.6.14 | 6-sulfate group of the N-acetyl- |
| sulfatase | | D-glucosamine 6-sulfate; |
| (Glucosamine-6-sulfatase; | | heparan sulfate; keratan sulfate. |
| Chondroitinsulfatase) | | |
| N-sulfoglucosamine-3- | 3.1.6.15 | 3-sulfate groups of the N-sulfo- |
| sulfatase | | D-glucosamine 3-O-sulfate |
| (Chondroitinsulfatase) | | residues of heparin; N-acetyl- |
| | | D-glucosamine 3-O-sulfate |
| Monomethyl-sulfatase | 3.1.6.16 | Monomethyl sulfate |
| D-lactate-2-sulfatase | 3.1.6.17 | (S)-2-O-sulfolactate |
| Glucuronate-2-sulfatase | 3.1.6.18 | 2-sulfate groups of the 2-O- |
| (Chondro-2-sulfatase) | | sulfo-D-glucuronate residues of |
| | | chondroitin sulfate, heparin and |
| | | heparitin sulfate. |

The sulfatase substrate can be designed to be reactive with a particular sulfatase or a group of sulfatases, or it can be designed to determine substrate specificity and other catalytic features, such as determining a value for kcat or Km. The sulphate ester in the sulfatase recognition moiety can be any group that is capable of being desulfated by a sulfatase.

In addition to having one or more sulphate esters capable of being desulfated, the sulfatase substrate moiety can include additional groups, for example amino acid residues (or analogs thereof) that facilitate binding specificity, affinity, and/or rate of desulfated by the sulfatase.

In some embodiments, a peptidase substrate for detecting, quantifying and/or characterizing one or more protein peptidases in a sample is provided. In other embodiments the peptide moiety can be designed to be reactive with a particular peptidase or group of peptidases. A peptidase is any member of a subclass of enzymes of the hydrolase class that catalyze the hydrolysis of peptide bonds. Generally, peptidases are divided into exopeptidases that act only near a terminus of a polypeptide chain and endopeptidases that act internally in polypeptide chains. The peptidase to be detected can be any peptidase known in the art. Also, the peptidase can be a peptidase candidate, and the methods used to confirm and/or characterize the peptidase activity of the candidate.

A wide variety of peptidases have been identified. Generally, peptidases are classified according to their catalytic mechanisms: 1) serine peptidases (such as such as chymotrypsin and trypsin); 2) cysteine peptidases (such as papain); 3) aspartic peptidases (such as pepsin); and, 4) metallo peptidases (such as thermolysin).

In some cases, peptidases are highly specific for only one or a few proteins, but in other cases, peptidases are relatively non-specific and can act on a large range of protein targets. Accordingly, compositions can be designed to detect particular peptidases by suitable selection of the peptidase substrate moiety. Exemplary peptidases and preferential cleavage sites, as indicated by “-|-” are shown in Table 6, below. These various cleavage sites can be used to design peptidase substrate moieties having desired specificities for particular peptidases and/or peptidase families.

TABLE 6

| Peptidase | EC number | Preferential cleavage |
|---|---|---|
| Chymotrypsin. | 3.4.21.1 | Tyr-\|-Xaa, Trp-\|-Xaa, Phe-\|- |
| | | Xaa, Leu-\|-Xaa |
| Trypsin | 3.4.21.4 | Arg-\|-Xaa, Lys-\|-Xaa. |
| Thrombin | 3.4.21.5 | Arg-\|-Gly |
| Renin | 3.4.23.15 | Pro-Phe-His-Leu-\|-Val-Ile |

Xaa - denotes any amino acid

The peptidase substrate moiety can be designed to be reactive with a particular peptidase or a group of peptidases, or it can be designed to determine substrate specificity and other catalytic features, such as determining a value for kcat or Km.

In addition to having one or more peptide bonds capable of being hydrolyzed, the peptidase substrate moiety can include additional amino acid residues (or analogs thereof) that facilitate binding specificity, affinity, and/or rate of hydrolysis by the peptidase.

5.8 Methods

The compositions described herein find a wide variety of uses in detecting, quantifying and/or characterizing enzymes and agents in biological, medical and industrial applications. The methods generally comprise detecting, quantifying and/or characterizing enzymes in a sample with a composition comprising (i) a hydrophobic molecule comprising a hydrophobic moiety, a dye moiety and an optional charge-moiety; and, (ii) one or more charge-balance molecules. In some embodiments, the charge-balance molecules can be the same. In some embodiments, the charge-balance molecules can be different. In some embodiments, the hydrophobic molecule and/or charge-balance molecule, can each independently of the other, comprise a substrate or putative substrate for enzymes or agents of interest. In some embodiments, the optional charge-moiety comprises an enzyme substrate. In some embodiments, the hydrophobic molecule and the charge-balance molecule both comprise the same substrate. In some embodiments, the hydrophobic molecule and the charge-balance molecule comprise different substrates.

In some embodiments, the method comprises the steps of (i) contacting a sample with a composition described herein, under conditions effective to permit the enzyme or agent, when present in the sample, to act on the substrate(s) in a manner that leads to an increase in a signal produced by the dye moiety; and (ii) detecting the signal, where an increase in the signal indicates the presence and/or quantity of the enzyme in the sample.

The sample to be tested can be any suitable sample selected by the user. The sample can be naturally occurring or man-made. For example, the sample can be a blood sample, tissue sample, cell sample, buccal sample, skin sample, urine sample, water sample, or soil sample. The sample can be from a living organism, such as a eukaryote, prokaryote, mammal, human, yeast, or bacterium. The sample can be processed prior to contact with a substrate of the present teachings by any method known in the art. For example, the sample can be subjected to a lysing step, precipitation step, column chromatography step, heat step, etc. In some cases, the sample is a purified or synthetically prepared enzyme that is used to screen for or characterize an enzyme substrate, inhibitor, activator, or modulator.

If the sample contains multiple enzymes, for example both a kinase and a phosphatase, so that the activity of one can interfere with the activity of the other, then an inactivating agent (e.g., an active site directed an irreversible inhibitor) can be added to the sample to inactivate whichever activity is not desired.

The reaction mixture typically includes a buffer, such as a buffer described in the “Biological Buffers” section of the 2000-2001 Sigma Catalog. Exemplary buffers include MES, MOPS, HEPES, Tris (Trizma), bicine, TAPS, CAPS, and the like. The buffer is present in an amount sufficient to generate and maintain a desired pH. The pH of the reaction mixture is selected according to the pH dependency of the activity of the enzyme to be detected, and the charge of the various moieties described herein. For example, the pH can be from 2 to 12, from 5 to 9, or from 6 to 8. The reaction mixture can also contains salts, reducing agents such as dithiothreitol (DTT), and any necessary cofactors and/or cosubstrates for the enzyme (e.g., ATP for a protein kinase, Ca²⁺ ion for a calcium dependent kinase, and cAMP for a protein kinase A). In one embodiment, the reaction mixture does not contain detergent or is substantially free from detergents.

In some embodiments, it can be desirable to dilute the sample to be tested to as low a concentration as reasonably possible to help avoid masking charged groups in the compositions described herein. The sample to be tested can be diluted to any concentration that permits a detectable increase in fluorescence. In some embodiments the sample can be diluted 1, 2, 5, 10, 20, 30, 40, or 50-fold. In some embodiments, a greater than 50-fold dilution of the sample can be desirable. In some embodiments, the sample can be diluted in the assay reaction mixture.

In some embodiments, it can be desirable to keep the ionic strength as low as reasonably possible to help avoid masking charged groups in the reaction product, so that micelle formation remains disfavored and destabilized. For example, high salt concentration (e.g., 1 M NaCl) can be inappropriate. In addition, it can be desirable to avoid high concentrations of certain other components in the reaction mixture that can also adversely affect the fluorescence properties of the product. Guidance regarding the effects of ionic species, such as metal ions, can be found in Surfactants and Interfacial Phenomena, 2nd Ed., M. J. Rosen, John Wiley & Sons, New York (1989), particularly chapter 3. For example, Mg²⁺ ion at a concentration of 5 mM is used in the Examples provided below, but higher concentrations can give poorer results.

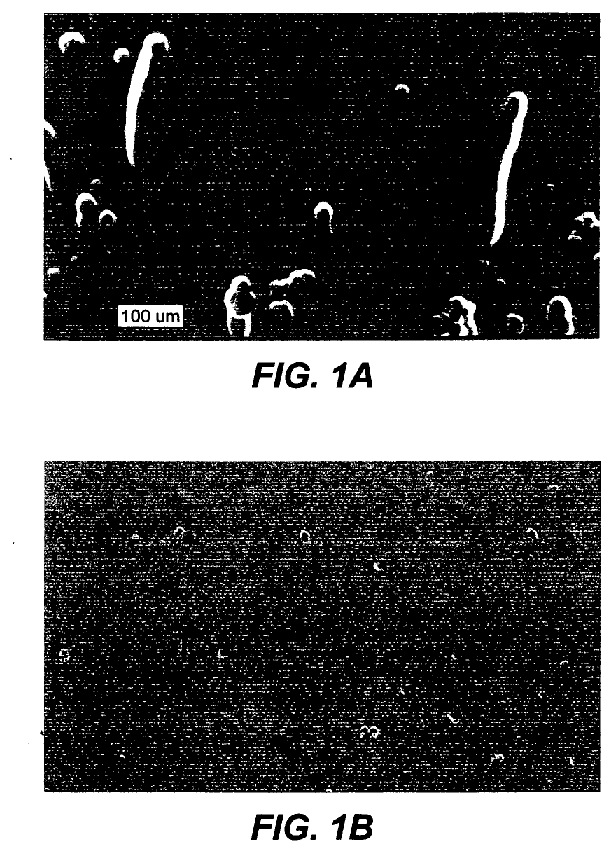

Micelle formation can be detected in a variety of ways, including fluorescence titration of the molecules in detergent, and dynamic laser light scattering. Additionally, direct visual evidence of micelle formation, and micelle disruption by adding a charged group, can be obtained by freeze fracturing electron microscopy. For example, FIG. 1A is an electron micrograph of micelles comprising the hydrophobic molecule, C₁₇OOOK(tet)RQGSFRA-amide. In the hydrophobic molecule, the hydrophobic moiety comprises a carbon chain (C₁₇), the dye moiety (tet) is linked to the hydrophobic moiety and an optional linker via the amino acid lysine (K). “Tet” is a fluorescent moiety provided by 2′,7′,4,7-tetachloro-5-carboxy fluorescein (2′,7′-dichloro-5-carboxy-4,7-dichlorofluorescein). OOO represents optional O-spacers comprising (bis)ethylene glycol group(s). FIG. 1A shows the hydrophobic molecule is capable of forming cylindrical or tubular micelles (200-1000 nm in length and 20-60 nm in diameter), clusters of spheres (5-20 micelles), and individual micelles. FIG. 1B is an electron micrograph of micelles comprising a phosphorylated hydrophobic molecule, C₁₇OOOK(tet)RQGS(p)FRA-amide. Phosphorylation of the hydrophobic molecule at the serine residue causes the addition of two negative charges, for a net change in charge of ⁻2. In contrast, to the tubular micelle formed by the dephosphorylated hydrophobic molecules, the phosphorylated hydrophobic molecules only form small spheres and small clusters of spheres (up to 5 micelles). These results show that dephosphorylated hydrophobic molecules form large aggregates of monomers and the phosphorylated hydrophobic molecules form smaller aggregates with few monomers. Thus, the addition of two negative charges to the hydrophobic molecule, results in micelle disruption and deaggregation.

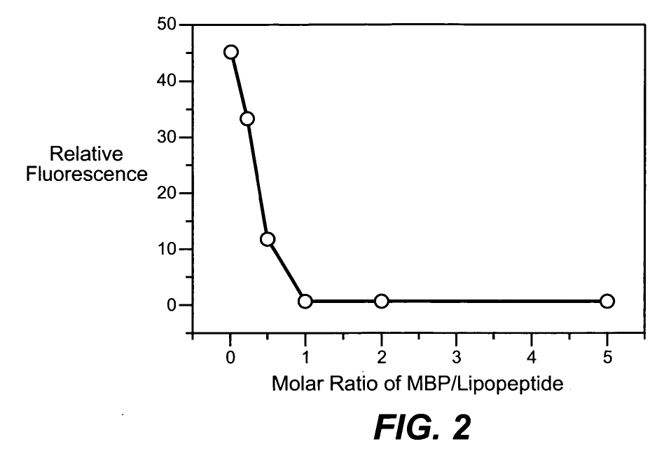

FIG. 2 is an exemplary embodiment showing the addition of positively charged MBP (+18) is capable of quenching the fluorescence of a hydrophobic molecule (−5), comprising C₁₆OOOK(Dye2)EEIYGEF-amide. The hydrophobic moiety is a C₁₆ carbon chain, the dye moiety (Dye2) is 5-carboxy-2′,7′-dipyridyl-sulfonefluorescein, linked via the optional amino acid lysine to the hydrophobic moiety, and is merely an exemplary linker. OOO represents an optional O-spacers. The hydrophobic molecule has a net negative charge of −5, wherein Dye2 has a charge of −2 and the charged group has a charge of −3. The relative fluorescence of the negatively charged hydrophobic molecule decreases as the concentration of positively charged MBP is increased in the solution. In FIG. 2, the addition of MPB to the hydrophobic molecule at a less than a 1:1 molar ratio promotes micelle formation, and thereby quenches the fluorescence of the fluorescence dye. The addition of MPB to the hydrophobic molecule at 1:1 molar ratio or above promotes micelle formation, and results in almost complete quenching of the fluorescence of the fluorescent dye. While not intending to be bound by any theory of operation, it is believed that the MPB comprises sufficient opposite charge from the hydrophobic molecule to promote or encourage micelle formation, thereby quenching the fluorescence of the dye moiety.

In practicing certain aspects of the methods, a hydrophobic molecule (or hydrophobic molecule and charge-balance molecule) is mixed with a sample containing an enzyme that is to be detected or that is being used to screen for, detect, quantify, and/or characterize a compound for substrate, inhibitor, activator, or modulator activity. Reaction of the enzyme with the substrate causes an increase (to a more charged species) in the absolute amplitude of the net charge of the micelle, such that the fluorescence of the reacted micelle is greater than the fluorescence of the unreacted micelle. In some embodiments, the reaction of the substrate with the enzyme makes the substrate either (1) net negatively charged by (1A) adding or generating a new negatively charged group on the substrate, or (1B) removing or blocking a positively charged group on the substrate; or (2) net positively charged, by (2A) adding or generating a new positively charged group on the substrate, or (2B) removing or blocking a negatively charged group on the substrate.

For example, reaction (1A) can be accomplished by adding a phosphate group to a hydroxyl group on the substrate (changing a neutrally charged group to a group having a charge of −2, (e.g., using a protein kinase), by cleaving a carboxylic ester or amide to produce a carboxyl group (changing a neutrally charged group to a group having a charge of −1, e.g., using an esterase or amidase). Reaction (1B) can be accomplished by cleaving positively charged amino acids, or can be accomplished by reacting an amino or hydrazine group in the enzyme recognition moiety with an acetylating enzyme to produce a neutral acetyl ester group, with an N-oxidase enzyme to produce a neutral N-oxide, with an ammonia lyase to remove ammonia, or with an oxidase that causes oxidative deamination, for example. Reaction (2A) can be accomplished, for example, by treating an amide group in the substrate with an amidase to generate a positively charged amino group in the substrate molecule. Reaction (2B) can be accomplished by cleaving negatively charged amino acids, or can be accomplished using a decarboxylase enzyme to remove a carboxylic acid, or by reacting a carboxyl group with a methyl transferase to form a carboxylic ester, for example. A variety of enzymes capable of performing such transformations are known in the literature (e.g., see C. Walsh, Enzymatic Reaction Mechanisms, WH Freeman and Co., New York, (1979), the Worthington Product Catalog (Worthington Enzymes), Sigma Life Sciences Catalog, and the product catalogs of other commercial enzyme suppliers).

While the basis for increased fluorescence is not certain, and the inventors do not wish to be bound to a particular theory, it is contemplated that the fluorescent substrate molecule and/or charge-balance molecule of the present teachings are capable of forming micelles in the reaction mixture due to the hydrophobic moiety(ies), so that the fluorescent dyes quench each other due to their close proximity. Micelle formation can be particularly favored when the charge on the substrate molecule is offset by the charge on the charge-balance molecule so that micelle formation is not prevented by mutual charge repulsion. While not intending to be bound by any theory of operation, it is believed that ionic bonds can be formed between oppositely charged charge-balance molecule and the substrate molecule in aqueous solution at physiological pH and promote or encourage micelle formation. For example, FIG. 2 shows that the addition of varying concentrations of charge-balance molecule, MBP, quenches the fluorescence of a hydrophobic molecule, C₁₆OOOK(Dye2)EEIYGEF (10 μM) in 25 mM Tris (pH 7.6). While not intending to be bound by any theory of operation, it is contemplated that the fluorescent hydrophobic molecule and charge-balance molecule are capable of forming micelles so that the fluorescent dyes quench each other due to their close proximity.

In some embodiments, the charge-moiety comprises the peptide E-E-I-Y-G-E-F-(SEQ ID NO:1) and has a net charge of about −3 at about pH 7.6. In some embodiments, the hydrophobic molecule comprise the structure C₁₆OOOK(Dye2) EEIYGEF-amide, wherein the hydrophobic moiety is a C₁₋₆ carbon chain, OOO represents the optional O—spacers, and Dye2 is 5-carboxy-2′,7′-dipyridyl-sulfonefluorescein. In this exemplary embodiment, the fluorescent moiety, 5-carboxy-2′,7′-dipyridyl-sulfonefluorescein is linked to the hydrophobic moiety and an optional linker via the amino acid lysine (K). As will be appreciated by a person of skill in the art, the illustrated lysine is merely an exemplary linker.

In some embodiments, the charge-moiety comprise the peptide K-K-A-A-G-K-L (SEQ ID NO:2) and has a net charge of about +3 at about pH 7.6. In some embodiments, the hydrophobic molecule comprises the structure C₁₆OOOK(Dye2)KKKKAAGKL-amide, wherein hydrophobic moiety is a C₁₋₆ carbon chain, OOO represents the optional O-spacers, and Dye2 is 5-carboxy-2′,7′-dipyridyl-sulfonefluorescein. In this exemplary embodiment, the fluorescent moiety, 5-carboxy-2′,7′-dipyridyl-sulfonefluorescein is linked to the hydrophobic moiety and an optional linker via the amino acid lysine (K). As will be appreciated by a person of skill in the art, the illustrated lysine is merely an exemplary linker.

To be effective, not only should a complex comprising a hydrophobic molecule and charge-balance molecule react with the enzyme to form the desired modified product, but the product should be more fluorescent than the compound comprising the hydrophobic molecule and charge-balance molecule, so that a detectable increase in fluorescence can be observed. Generally, a greater change in fluorescence provides greater assay sensitivity, provided that an adequately low signal-to-noise ratio is achieved. Therefore, it can be desirable to test multiple hydrophobic molecules and a charge-balance molecules to find a complex having the most suitable fluorescence properties.

The compositions described herein are useful in the detection of enzymes. Real-time kinase assays for PKCβII, MAP kinase1/Erk1, and MAP kinase2/Erk2 using the hydrophobic molecule, C₁₆OOOK(Dye 2)EEIYGEF-amide and charge-balance molecule, myelin basic protein with 0 or 100 μM ATP, are shown in FIGS. 3A-C. The addition of the enzyme to the micelle comprising the hydrophobic molecule and charge-balance molecule causes a greater than 4 fold increase in fluorescence over time.

The present disclosure contemplates not only detecting enzymes, but also methods involving: (1) screening for and/or quantifying enzyme activity in a sample, (2) determining kcat and/or Km of an enzyme or enzyme mixture with respect to selected substrates, (3) detecting, screening for, and/or characterizing substrates of enzymes, (4) detecting, screening for, and/or characterizing inhibitors, activators, and/or modulators of enzyme activity, and (5) determining substrate specificities and/or substrate consensus sequences or substrate consensus structures for selected enzymes.

For example, in screening for enzyme activity, a sample that contains, or can contain, a particular enzyme activity is mixed with a substrate of the present teachings, and the fluorescence is measured to determine whether an increase in fluorescence has occurred. Screening can be performed on numerous samples simultaneously in a multi-well or multi-reaction plate or device to increase the rate of throughput. Kcat and Km can be determined by standard methods, as described, for example, in Fersht, Enzyme Structure and Mechanism, 2nd Edition, W.H. Freeman and Co., New York, (1985)).

In some embodiments, the reaction mixture can contain two or more different enzymes. This can be useful, for example, to screen multiple enzymes simultaneously to determine if an enzyme has a particular enzyme activity.

The substrate specificity of an enzyme can be determined by reacting an enzyme with different substrate molecules having different substrate moieties, and the activity of the enzyme toward the substrates can be determined based on an increase in fluorescence. For example, by reacting an enzyme with several different substrate molecules having several different protein kinase recognition moieties, a consensus sequence for a preferred substrate of a kinase can be prepared.

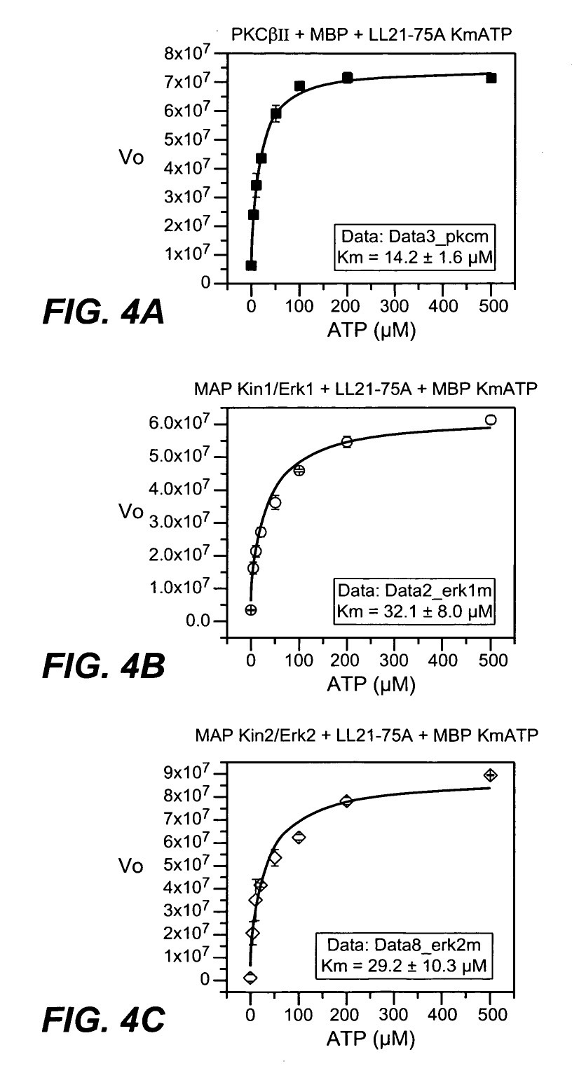

In some embodiments, the compositions described herein are useful in characterizing an enzyme's Kₘ^{ATP}. The Kₘ^{ATP} for PKCβII, MAP kinase1/Erk1, and MAP kinase2/Erk2 using the hydrophobic molecule, C₁₆OOOK(Dye 2)EEIYGEF-amide and charge-balance molecule, myelin basic protein, with increasing concentrations of with 0-500 μM ATP, are shown in FIGS. 4A-C. The addition of increasing concentrations of ATP to the micelle comprising the hydrophobic molecule and charge-balance molecule causes an increase in fluorescence. The apparent Kₘ^{ATP} for PKCβII, MAP kinase1/Erk1, and MAP kinase2/Erk2 are show in FIGS. 4A-C, respectively.

Although not necessary for operation of the methods, the assay mixture can optionally include one or more quenching moieties or quenching molecules designed to quench the fluorescence of the fluorescent moiety of the hydrophobic molecule and/or charge-balance molecule.

Detecting, screening for, and/or characterizing inhibitors, activators, and/or modulators of enzyme activity can be performed by forming reaction mixtures containing such known or potential inhibitors, activators, and/or modulators and determining the extent of increase or decrease (if any) in fluorescence signal relative to the signal that is observed without the inhibitor, activator, or modulator. Different amounts of these substances can be tested to determine parameters such as Ki (inhibition constant), K_{H} (Hill coefficient), Kd (dissociation constant) and the like to characterize the concentration dependence of the effect that such substances have on enzyme activity.

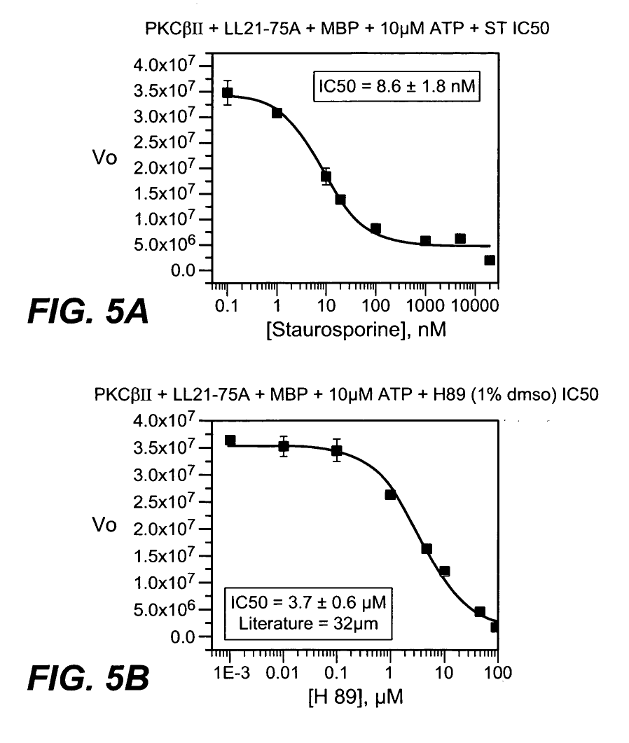

In some embodiments, the compositions described herein are useful in characterizing enzyme inhibitors. The IC₅₀ of staurosporine and H98 for PKCβII using the hydrophobic molecule, C₁₆OOOK(Dye 2)EEIYGEF-amide and charge-balance molecule, myelin basic protein, are shown in FIGS. 5A-B. The addition of increasing concentrations of the enzyme inhibitor to the micelle comprising the hydrophobic molecule and charge-balance molecule causes a decrease in fluorescence. The apparent IC₅₀ of staurosporine and H98 for PKCβII are show in FIGS. 5A-B, respectively.

Detection of fluorescent signal can be performed in any appropriate way. Advantageously, substrate molecules/charge-balance molecules of the present teachings can be used in a continuous monitoring phase, in real time, to allow the user to rapidly determine whether enzyme activity is present in the sample, and optionally, the amount or specific activity of the enzyme. The fluorescent signal is measured from at least two different time points, usually until an initial velocity (rate) can be determined. The signal can be monitored continuously or at several selected time points. Alternatively, the fluorescent signal can be measured in an end-point embodiment in which a signal is measured after a certain amount of time, and the signal is compared against a control signal (before start of the reaction), threshold signal, or standard curve.

5.9 Kits

Also provided are kits for performing methods of the present teachings. In some embodiments, the kits comprise (i) a hydrophobic molecule comprising a hydrophobic moiety and an optional charge-moiety, and (ii) one or more charge-balance molecules. The hydrophobic molecule and/or charge-balance molecule comprises a dye moiety. In some embodiments, the hydrophobic molecule and/or charge-balance molecule can independently of the other comprise a substrate or putative substrate for enzymes or agents of interest. In some embodiments, the optional charge-moiety comprises an enzyme substrate. In some embodiments, the hydrophobic molecule and the charge-balance molecule both comprise the same substrate. In some embodiments, the hydrophobic molecule and the charge-balance molecule comprise different substrates.

In some embodiments, the kits comprise a hydrophobic molecule comprising a hydrophobic moiety and a dye moiety. In some embodiments, the kits comprise a hydrophobic molecule comprising a hydrophobic moiety, a charge-moiety, and a dye moiety. In some embodiments, the hydrophobic molecule comprises an enzyme substrate. In some embodiments, the charge-moiety comprises an enzyme substrate. In some embodiment, the kit further comprises a charge-balance molecule. In some embodiments, the charge balance molecule comprises a metal ion, charged oligonucleotide, charged oligonucleotide analog, oligonucleotide mimic, charged amino acid, charged peptide, or charged protein. In some embodiments, the kit comprises a charge-balance molecule comprising an enzyme substrate. In some embodiments, the hydrophobic molecule and the charge-balance molecule both comprise the same substrate. In some embodiments, the hydrophobic molecule and the charge-balance molecule comprise different substrates.

The kit may optionally comprise a quenching moiety and/or quenching molecule. The kit may optionally comprise additional components for making micelles. In some embodiments, the kit further comprises a buffer for preparing a reaction mixture that facilitates an enzyme reaction. The buffer can be provided in a container in dry form or liquid form. The choice of a particular buffer can depend on various factors, such as the pH optimum for the enzyme to be detected, the solubility and fluorescence properties of the fluorescent moiety in the substrate molecule and/or charge-balance molecule, and the pH of the sample from which the target enzyme is obtained. The buffer is usually added to the reaction mixture in an amount sufficient to produce a particular pH in the mixture. In some embodiments, the buffer is provided as a stock solution having a pre-selected pH and buffer concentration. Upon mixture with the sample, the buffer produces a final pH that is suitable for the enzyme assay, as discussed above. The pH of the reaction mixture can also be titrated with acid or base to reach a final, desired pH. The kit can additionally include other components that are beneficial to enzyme activity, such as salts (e.g., KCl, NaCl, or NaOAc), metal salts (e.g., Ca2+ salts such as CaCl₂, MgCl₂, MnCl₂, ZnCl₂, or Zn(OAc), detergents (e.g., TWEEN 20), and/or other components that can be useful for a particular enzyme. These other components can be provided separately from each other or mixed together in dry or liquid form.

The hydrophobic molecule and/or the charge-balance molecule can be provided in dry or liquid form, together with or separate from the buffer. To facilitate dissolution in the reaction mixture, the hydrophobic molecule and/or charge-balance molecule can be provided in an aqueous solution, partially aqueous solution, or non-aqueous stock solution that is miscible with the other components of the reaction mixture. For example, in addition to water, a substrate solution can also contain a cosolvent such as dimethyl formamide, dimethylsulfonate, methanol or ethanol, typically in a range of 1%-10% (v:v).

The kit can also contain additional chemicals useful in the detection, quantifying, and/or characterizing of enzymes. For example, for the detection of protein kinase activity, the kit can also contain a phosphate-donating group, such as ATP, GTP, ITP (inosine triphosphate) or other nucleotide triphosphate or nucleotide triphosphate analogs that can be used by the kinase to phosphorylate the substrate moiety.

The operation of the various compositions and methods can be further understood in light of the following non-limiting examples that illustrate various aspects of the present teachings, which should not be construed as limiting the scope of the present teachings in any way.

### EXAMPLES

6.1 Cryoelectron Microsopy of Micelles

For freeze fracture electron microscopy, the hydrophobic molecule, C₁₇OOOK(tet)RQGSFRA-amide phosphorylated hydrophobic molecule C₁₇OOOK (tet)RQGS(p)FRA-amide were each dissolved in 25 mM Tris (pH 7.6), 5 mM MgCl and 5 mM DTT. The samples were frozen in liquid nitrogen-cooled propane. The cooling rate of 10,000 Kelvin/second was achieved to avoid ice crystals formation and artifacts possibly caused by the cryofixation processing. The cryofixed samples were stored in liquid nitrogen for less than two hours before possessing. The fracturing process was carried out in a JEOL JED-9000 freeze-etching machine and the exposed fractured planes were shadowed with Pt for thirty seconds at an angle of 25-35C and with carbon for 35 seconds (2 kV/60-70 mA, 1×10⁻⁵ Torr). The replicas produced were cleaned with concentrated chloroform/methanol (1:1 by volume) at least five times. The cleaned replicas were examined with a JEOL 1000CX or Philips CM 10 electron microscope.

6.2 Addition of Charge-Balance Molecule Quenches the Fluorescence of the Hydrophobic Molecule

A reaction solution was prepared containing 10 μM hydrophobic molecule C₁₆OOOK(Dye2)EEIYGEF-amide and 25 mM Tris (pH 7.6), 5 mM MgCl and 5 mM DTT. Varying concentrations of the charge-balance molecule, Myelin Basic Protein (Upstate USA, Inc. cat. no: 13-104) were added (final concentration 0, 2.5, 5, 10, 20, and 50 μM) and the fluorescence was determined. The results are shown in FIG. 2.

6.3 Detection of Protein Kinase Activity

A reaction solution (10 μl) was prepared containing the hydrophobic molecule C₁₆OOOK(Dye2)EEIYGEF-amide (10 μM), and 10 μM charge-balance molecule Myelin Basic Protein (Upstate cat. no: 13-104), in 20 mM Tris buffer, pH 7.6, MgCl₂ (5 mM), DTT (5 mM) and either PKCβII (0.15 ng/μl, Upstate USA, Inc.), MAP kinase1/Erk1 (1.5 ng/μl, Upstate USA, Inc.), or MAP kinase2/Erk2 (1.5 ng/μl, Upstate USA, Inc.). The solution was pipetted into wells of a 384-well plate (10 μL per well), Corning 384-well, black, non-binding surface (NBS), microwell plates. ATP (0 or 500 μM) was added to initiate the kinase reaction. Fluorescence was read in real-time every 2 minutes for 2 hours, at ambient temperature, using, Molecular Devices (Sunnyvale, Calif.) Analyst GT, with excitation and emission set at 485 and 535 nm respectively. The results for PKCβII, MAP kinase1/Erk1, and MAP kinase2/Erk2 are shown in FIGS. 3A-C, respectively.

6.4 Kₘ^{ATP} of Protein Kinases

Real-time kinase assays was used to determine the apparent Kₘ^{ATP} for several protein kinases. A reaction solution (10 μl) was prepared containing the hydrophobic molecule C₁₆OOOK(Dye2)EEIYGEF-amide (10 μM), and 10 μM charge-balance molecule Myelin Basic Protein (Upstate USA, Inc. cat. no: 13-104), in 20 mM Tris buffer, pH 7.6, MgCl₂ (5 mM), DTT (5 mM), 10% Lipid Activator (Upstate USA, Inc), and either PKCβII (0.15 ng/μl, Upstate USA, Inc.), MAP kinase1/Erk1 (1.5 ng/μl, Upstate USA, Inc.), or MAP kinase2/Erk2 (1.5 ng/μl, Upstate USA, Inc.). The solution was pipetted into wells of a 384-well plate (9 μL per well), Corning 384-well, black, non-binding surface (NBS), microwell plates. ATP (1 μL) at eight different concentrations (0, 5, 10, 20, 50, 100, 200, or 500 μM) was added to initiate the kinase reaction. Fluorescence was read in real-time every 2 minutes for 2 hours, at ambient temperature, using Molecular Devices (Sunnyvale, Calif.) Analyst GT, with excitation and emission set at 485 and 535 nm respectively. The initial velocity was fitted to Michaelis-Menton equation with the non-linear fitting program Origin 6.1 (OriginLab, MA). The results for PKCβII, MAP kinase1/Erk1, and MAP kinase2/Erk2 are shown in FIGS. 4A-C, respectively.

6.5 IC₅₀ of Staurosporine and H89 for PKCβII

A reaction solution (10 μl) was prepared containing the hydrophobic molecule C₁₆OOOK(Dye2)EEIYGEF-amide (10 μM), and 10 μM charge-balance molecule Myelin Basic Protein (Upstate USA, Inc. cat. no: 13-104), in 20 mM Tris buffer, pH 7.6, MgCl₂ (5 mM), DTT (5 mM), 10% Lipid Activator (Upstate USA, Inc), and PKCβII (0.15 ng/μl, Upstate USA, Inc.). The solution was pipetted into wells of a 384-well plate (10 μL per well), Corning 384-well, black, non-binding surface (NBS), microwell plates. The enzyme inhibitor staurosporine (Sigma) at eight different concentrations (0.1, 1, 10, 20, 100, 1000, 5000, or 20000 nM) or H89 (Sigma) at eight different concentrations (0.001, 0.01, 1, 5, 10, 50, or 100 μM) in final concentration of 1% DMSO was added. ATP (10 μM) was added to initiate the kinase reaction. Fluorescence was read in real-time every 2 minutes for 2 hours, at ambient temperature, using, Molecular Devices (Sunnyvale, Calif.) Analyst GT, with excitation and emission set at 485 and 535 nm respectively. The results for staurosporine and H89 are shown in FIGS. 4A-C, respectively.

All publications and patent applications mentioned herein are hereby incorporated by reference as if each publication or patent application was specifically and individually indicated to be incorporated by reference.

The section headings used herein are for organizational purposes only and are not to be construed as limiting the subject matter described in any way.

While the present teachings are described in conjunction with various embodiments, it is not intended that the present teachings be limited to such embodiments. On the contrary, the present teachings encompass various alternatives, modifications, and equivalents, as will be appreciated by those skilled in the art. While various specific embodiments have been illustrated and described, it will be appreciated that various changes can be made without departing from the spirit and scope of the invention(s).

1. A micelle comprising (i) a hydrophobic molecule comprising a hydrophobic moiety capable of integrating the hydrophobic molecule into the micelle, a dye moiety and an optional charge-moiety; and, (ii) one or more charge-balance molecules capable of promoting micelle formation at physiological pH; wherein the hydrophobic molecule and/or the charge-balance molecules comprise an enzyme substrate.

2. The micelle of claim 1 in which the hydrophobic molecule is negatively charged and the charge-balance molecule is positively charged.

3. The micelle of claim 1 in which the hydrophobic molecule is positively charged and charge-balance molecule is negatively charged.

4-19. (canceled)

20. The micelle of claim 1 in which the charge-balance molecule is a charged protein, wherein the concentration of the charged protein is about 2 times-greater than the concentration of an endogenous charged protein in a sample.

21. The micelle of claim 20 in which said charged protein and said endogenous charged protein are the same protein.

22. The micelle of claim 20 in which said charged protein and said endogenous charged protein are different proteins.

23. The micelle of claim 20 in which the charged protein is selected from a myelin basic protein, myelin P2 protein, casein and any combination thereof.

24. The micelle of claim 20 in which the charged protein is a myelin basic protein.

25. The micelle of claim 24 in which charge-moiety comprises the sequence E-E-I-Y-G-E-F (SEQ ID NO:1).

26. The micelle of claim 1 in which the hydrophobic molecule comprises the enzyme substrate.

27. The micelle of claim 1 in which the charge-balance molecule comprises the enzyme substrate.

28. The micelle of claim 1 in which the hydrophobic molecule and the charge-balance molecule each independently of the other comprise an enzyme substrate.

29-30. (canceled)

31. The micelle of claim 1 in which the enzyme is selected from a kinase, phosphatase, sulfatase, peptidase, carboxylase and any combination thereof.

32. The micelle of claim 31 in which the enzyme is a kinase.

33. The micelle of claim 32 in which the kinase is selected from PKA, PKC, MAPK, calmodulin-dependent protein kinase, phosphorylase kinase, Rafl, MEK, MEKK and any combination thereof.

34. The micelle of claim 1 in which the hydrophobic moiety comprises a hydrocarbon containing from 6 to 30 carbon atoms.

35. (canceled)

36. The micelle of claim 1 in which the dye moiety is a fluorescent moiety.

37-39. (canceled)

40. The micelle of claim 1 further comprising a quenching molecule comprising a hydrophobic moiety and a quenching moiety capable of quenching the fluorescence of a fluorescent moiety.

41-43. (canceled)

44. A method of detecting and/or characterizing an enzyme activity in a sample, comprising the steps of:
(i) contacting the sample with a micelle according to claim 1, under conditions effective to permit the enzyme, when present in the sample, to act on the substrate(s) in a manner that leads to an increase in a signal produced by the dye moiety; and
(ii) detecting the signal, where an increase in the signal indicates the presence and/or quantity of the enzyme in the sample.

45. A kit for detecting and/or characterizing an enzyme activity in a sample comprising (i) a hydrophobic molecule comprising a hydrophobic moiety capable of integrating the hydrophobic molecule into the micelle, a dye moiety and an optional charge-moiety, and (ii) a one or more charge-balance molecules; wherein the hydrophobic molecule and/or charge-balance molecule(s) comprise and an enzyme substrate.

46-56. (canceled)


SEQUENCE LISTING (39 sequences)

SEQ ID NO: 1 - protein, 7 aa, Artificial Sequence
  Description of Artificial Sequence Synthetic peptide
EEIYGEF

SEQ ID NO: 2 - protein, 7 aa, Artificial Sequence
  Description of Artificial Sequence Synthetic peptide
KKAAGKL

SEQ ID NO: 3 - protein, 5 aa, Artificial Sequence
  Description of Artificial Sequence Synthetic peptide
RRXXX

SEQ ID NO: 4 - protein, 7 aa, Artificial Sequence
  Description of Artificial Sequence Synthetic peptide
LRRASLG

SEQ ID NO: 5 - protein, 6 aa, Artificial Sequence
  Description of Artificial Sequence Synthetic peptide
RXXXFF

SEQ ID NO: 6 - protein, 7 aa, Artificial Sequence
  Description of Artificial Sequence Synthetic peptide
RQGSFRA

SEQ ID NO: 7 - protein, 4 aa, Artificial Sequence
  Description of Artificial Sequence Synthetic peptide
XPXX

SEQ ID NO: 8 - protein, 4 aa, Artificial Sequence
  Description of Artificial Sequence Synthetic peptide
PXXP

SEQ ID NO: 9 - protein, 9 aa, Artificial Sequence
  Description of Artificial Sequence Synthetic peptide
KKKKRFSFK

SEQ ID NO: 10 - protein, 8 aa, Artificial Sequence
  Description of Artificial Sequence Synthetic peptide
XRXXSXRX

SEQ ID NO: 11 - protein, 9 aa, Artificial Sequence
  Description of Artificial Sequence Synthetic peptide
LRRLSDSNF

SEQ ID NO: 12 - protein, 10 aa, Artificial Sequence
  Description of Artificial Sequence Synthetic peptide
KKLNRTLTVA

SEQ ID NO: 13 - protein, 7 aa, Artificial Sequence
  Description of Artificial Sequence Synthetic peptide
EEIYXXF

SEQ ID NO: 14 - protein, 8 aa, Artificial Sequence
  Description of Artificial Sequence Synthetic peptide
EEIYGEFR

SEQ ID NO: 15 - protein, 6 aa, Artificial Sequence
  Description of Artificial Sequence Synthetic peptide
EIYEXX

SEQ ID NO: 16 - protein, 6 aa, Artificial Sequence
  Description of Artificial Sequence Synthetic peptide
IYMFFF

SEQ ID NO: 17 - protein, 4 aa, Artificial Sequence
  Description of Artificial Sequence Synthetic peptide
YMMM

SEQ ID NO: 18 - protein, 5 aa, Artificial Sequence
  Description of Artificial Sequence Synthetic peptide
EEEYF

SEQ ID NO: 19 - protein, 12 aa, Artificial Sequence
  Description of Artificial Sequence Synthetic peptide
RIGEGTYGVVRR

SEQ ID NO: 20 - protein, 7 aa, Artificial Sequence
  Description of Artificial Sequence Synthetic peptide
RPRTSSF

SEQ ID NO: 21 - protein, 7 aa, Artificial Sequence
  Description of Artificial Sequence Synthetic peptide
PRTPGGR

SEQ ID NO: 22 - protein, 8 aa, Artificial Sequence
  Description of Artificial Sequence Synthetic peptide
RLNRTLSV

SEQ ID NO: 23 - protein, 8 aa, Artificial Sequence
  Description of Artificial Sequence Synthetic peptide
DRRLSSLR

SEQ ID NO: 24 - protein, 12 aa, Artificial Sequence
  Description of Artificial Sequence Synthetic peptide
EAIYAAPFARRR

SEQ ID NO: 25 - protein, 8 aa, Artificial Sequence
  Description of Artificial Sequence Synthetic peptide
EEIYGEFR

SEQ ID NO: 26 - protein, 15 aa, Artificial Sequence
  Description of Artificial Sequence Synthetic peptide
KVEKIGEGTYGVVYK

SEQ ID NO: 27 - protein, 8 aa, Artificial Sequence
  Description of Artificial Sequence Synthetic peptide
EEIYGEFR

SEQ ID NO: 28 - protein, 12 aa, Artificial Sequence
  Description of Artificial Sequence Synthetic peptide
RHSSPHQSEDEE

SEQ ID NO: 29 - protein, 18 aa, Artificial Sequence
  Description of Artificial Sequence Synthetic peptide
RRKDLHDDEEDEAMSITA

SEQ ID NO: 30 - protein, 4 aa, Artificial Sequence
  Description of Artificial Sequence Synthetic peptide
SXXX

SEQ ID NO: 31 - protein, 4 aa, Artificial Sequence
  Description of Artificial Sequence Synthetic peptide
SXXX

SEQ ID NO: 32 - protein, 17 aa, Artificial Sequence
  Description of Artificial Sequence Synthetic peptide
KGPWLEEEEEAYGWLDF

SEQ ID NO: 33 - protein, 9 aa, Artificial Sequence
  Description of Artificial Sequence Synthetic peptide
KKKKAAGKL

SEQ ID NO: 34 - protein, 7 aa, Artificial Sequence
  Description of Artificial Sequence Synthetic peptide
RQGSFRA

SEQ ID NO: 35 - protein, 7 aa, Artificial Sequence
  Description of Artificial Sequence Synthetic peptide
RRIPLSP

SEQ ID NO: 36 - protein, 8 aa, Artificial Sequence
  Description of Artificial Sequence Synthetic peptide
EEEIYGEF

SEQ ID NO: 37 - protein, 10 aa, Artificial Sequence
  Description of Artificial Sequence Synthetic peptide
RRRDDDSDDD

SEQ ID NO: 38 - protein, 7 aa, Artificial Sequence
  Description of Artificial Sequence Synthetic peptide
PXSPXSP

SEQ ID NO: 39 - protein, 6 aa, Artificial Sequence
  Description of Artificial Sequence Synthetic peptide
PFHLVI